SEQUENCE LISTING (32 sequences)

SEQ ID NO: 1 - RNA, 22 nt, Homo sapiens
uuuguucguucggcucgcguga

SEQ ID NO: 2 - RNA, 24 nt, Homo sapiens
uggaagacuagugauuuuguuguu

SEQ ID NO: 3 - RNA, 22 nt, Homo sapiens
uacccuguagaaccgaauuugu

SEQ ID NO: 4 - RNA, 23 nt, Homo sapiens
uaacacugucugguaacgauguu

SEQ ID NO: 5 - RNA, 24 nt, Homo sapiens
uguaaacauccucgacuggaagcu

SEQ ID NO: 6 - RNA, 22 nt, Homo sapiens
uacccuguagauccgaauuugu

SEQ ID NO: 7 - RNA, 22 nt, Homo sapiens
uaacagucuccagucacggcca

SEQ ID NO: 8 - RNA, 22 nt, Homo sapiens
uagcagcacaucaugguuuaca

SEQ ID NO: 9 - RNA, 23 nt, Homo sapiens
uacccauugcauaucggaguugu

SEQ ID NO: 10 - RNA, 21 nt, Homo sapiens
uagcaccaucugaaaucgguu

SEQ ID NO: 11 - RNA, 22 nt, Homo sapiens
ucaagagcaauaacgaaaaaug

SEQ ID NO: 12 - RNA, 24 nt, Homo sapiens
agcuacaucuggcuacugggucuc

SEQ ID NO: 13 - RNA, 20 nt, Homo sapiens
cggcucugggucugugggga

SEQ ID NO: 14 - RNA, 21 nt, Homo sapiens
gugaggacucgggagguggag

SEQ ID NO: 15 - RNA, 23 nt, Homo sapiens
ucuacagugcacgugucuccagu

SEQ ID NO: 16 - RNA, 22 nt, Homo sapiens
uccuucauuccaccggagucug

SEQ ID NO: 17 - RNA, 23 nt, Homo sapiens
ucuuugguuaucuagcuguauga

SEQ ID NO: 18 - RNA, 23 nt, Homo sapiens
caaagugcuguucgugcagguag

SEQ ID NO: 19 - RNA, 21 nt, Homo sapiens
ugguagacuauggaacguagg

SEQ ID NO: 20 - RNA, 21 nt, Homo sapiens
uccgagccugggucucccucu

SEQ ID NO: 21 - RNA, 22 nt, Homo sapiens
uauugcacucgucccggccucc

SEQ ID NO: 22 - RNA, 22 nt, Homo sapiens
ucccugagacccuaacuuguga

SEQ ID NO: 23 - RNA, 21 nt, Homo sapiens
cugaccuaugaauugacagcc

SEQ ID NO: 24 - RNA, 23 nt, Homo sapiens
ucuggcuccgugucuucacuccc

SEQ ID NO: 25 - RNA, 22 nt, Homo sapiens
uaauacugucugguaaaaccgu

SEQ ID NO: 26 - RNA, 22 nt, Homo sapiens
aaucguacagggucauccacuu

SEQ ID NO: 27 - RNA, 23 nt, Homo sapiens
uaaggugcaucuagugcagauag

SEQ ID NO: 28 - RNA, 23 nt, Homo sapiens
uuaaugcuaaucgugauaggggu

SEQ ID NO: 29 - RNA, 23 nt, Homo sapiens
uagcuuaucagacugauguugac

SEQ ID NO: 30 - RNA, 22 nt, Homo sapiens
aaaccguuaccauuacugaguu

SEQ ID NO: 31 - RNA, 22 nt, Homo sapiens
uccuguacugagcugccccgag

SEQ ID NO: 32 - RNA, 21 nt, Arabidopsis thaliana
uuuggauugaagggagcucua

(19) United States
(12) Patent Application Publication
(10) Pub. No.: US 2023/0272480 A1
(43) Pub. Date: Aug. 31, 2023

(54) Methods for Classifying Cancer

(71) Applicants: Queen's University at Kingston, Kingston (CA); Kingston Health Sciences Centre, Kingston (CA)

(72) Inventors: Neil Renwick, Kingston (CA); Kathrin Tyryshkin, Kingston (CA); Jina Nanayakkara, Kingston (CA); Xiaojing Yang, Kingston (CA)

(21) Appl. No.: 18/015,989

(22) PCT Filed: Jul. 15, 2021
(86) PCT No.: PCT/CA2021/050982

§ 371 Date: Jan. 13, 2023

Related U.S. Application Data
(60) Provisional application No. 63/052,267, filed on Jul. 15, 2020.

(51) Int. Cl. C12Q 1/6886; G16B 40/00

(52) U.S. Cl. CPC C12Q 1/6886; G16B 40/00; C12Q 2600/158; C12Q 2600/178; C12Q 2600/118; C12Q 2600/112

(57) ABSTRACT

Methods for classifying neuroendocrine neoplasms (NENs) are based on comprehensive microRNA (miRNA) expression profiling and data mining of multiple pathological types. Reference miRNA expression profiles are generated for multiple NEN pathological types and site-matched non-NEN controls, and candidate category and type specific miRNAs are identified and used for 5 classification. A multilayer hierarchical classifier for discriminating NEN pathological types is based on the candidate category and type specific miRNAs. Methods and software products include products that enable construction of discriminator functions including hierarchical classifiers for discriminating among multiple conditions of interest in a dataset, the methods and software products being applicable to a wide range of data modalities, including, for example, omits data 0 such as miRNA expression data wherein multiple conditions of interest include different cancer pathologies.

### FIELD

The invention relates to methods for classifying cancer. More specifically, methods relate to classifying cancer such as neuroendocrine neoplasms (NENs) based on microRNA (miRNA) expression profiling and use of a hierarchical classifier to discriminate among multiple NEN pathological types. The invention also relates to software products for multilayer hierarchical classifiers for discriminating among multiple conditions of interest in a dataset, the methods and software products being applicable to a wide range of data modalities.

### BACKGROUND

Classifying neuroendocrine neoplasms (NENs) is challenging due to tumor diversity, inconsistent terminology, and piecemeal molecular characterization. Currently, NENs are broadly divided into epithelial or non-epithelial groups based on site of origin and differences in keratin and other gene expression; each group comprising multiple pathological types (i.e., tumor types). To facilitate comparisons between NENs from different anatomical sites, a common classification framework has been proposed in which the terms ‘category’, ‘family’, ‘type’ and ‘grade’, respectively, denote predominant neuroendocrine differentiation, degree of differentiation, diagnostic entity, and inherent biological activity [1]. While morphological assessment and immunohistochemical staining for chromogranin A, synaptophysin, and Ki-67 proteins remain indispensable for confirming neuroendocrine differentiation and assessing tumor grade, other relevant molecular findings will be integrated into this framework over time. Such studies will help to answer many questions in NEN biology, including delineating the molecular differences between well-differentiated neuroendocrine tumors (NETs) and poorly differentiated neuroendocrine carcinomas (NECs) and finding regulatory molecules.

MicroRNAs (miRNAs) are small (e.g., 19-24 nt) regulatory RNA molecules that can also be used to classify cancer. miRNAs are highly informative tissue markers because of their abundance, cell-type and disease-stage specificity, and stability in fresh and archived materials such as tissues and biofluids. These molecules also provide valuable mechanistic insights into cellular processes due to computationally predictable interactions with messenger RNAs (mRNAs). In addition, miRNA expression profiles can be used to assess data reliability and to prioritize mRNA targets through further organization into miRNA cluster and sequence family datasets. To date, multiple miRNA profiling studies have been performed on single or limited combinations of NEN pathological types using different RNA isolation, detection, and analysis methods. The differences in approaches complicate interstudy comparisons, limiting the extent to biological and clinically relevant insights into NEN biology can be gained.

### SUMMARY

One aspect of the invention relates to a method for classifying cancer, comprising: obtaining data relating to expression levels of at least two selected miRNAs (miRs) in a biological sample from a subject; using a processor to subject the data to a discriminator function; wherein the discriminator function uses at least one selected feature in the data, the at least one selected feature being related to at least a first condition of interest; wherein the discriminator function comprises at least one trained classifier that classifies the cancer according to at least a first condition of interest based the at least one selected feature in the data.

In one embodiment the discriminator function comprises at least one classifier trained to use at least two selected features comprising expression levels of miR-375 and miR-7; wherein the at least one trained classifier classifies the cancer as a neuroendocrine neoplasm (NEN) or a non-neuroendocrine neoplasm (non-NEN).

In one embodiment the discriminator function comprises at least one classifier trained to use at least two selected features comprising expression levels of miR-200a and miR-10b; wherein the at least one trained classifier classifies the cancer according to a condition of interest as an epithelial NEN based on elevated expression of miR-200a and reduced expression of miR-10b, otherwise the at least one trained classifier classifies the cancer as a non-NEN.

In one embodiment the discriminator function comprises at least one classifier trained to use at least a selected feature comprising an expression level of miR-30a; wherein the at least one trained classifier classifies the NEN according to a condition of interest as parathyroid adenoma (PTA) based on elevated expression of miR-30a, otherwise the at least one trained classifier classifies the epithelial NEN as non-PTA.

In one embodiment the discriminator function comprises at least one classifier trained to use at least two selected features comprising expression levels of miR-10a and miR-212-3p; wherein the at least one trained classifier classifies the epithelial non-PTA NEN according to a condition of interest as pituitary adenoma (PitNET) based on reduced expression of miR-10a and elevated expression of miR-212-3p, otherwise the at least one trained classifier classifies the epithelial neuroendocrine neoplasm as non-PitNET.

In one embodiment the discriminator function comprises at least one classifier trained to use at least two selected features comprising expression levels of miR-15b and miR-660; wherein the at least one trained classifier classifies the epithelial non-PitNET NEN according to a condition of interest as Merkel cell carcinoma (MCC) based on elevated expression of miR-15b and miR-660, otherwise the at least one trained classifier classifies the epithelial NEN as non-MCC.

In one embodiment the discriminator function comprises at least one classifier trained to use at least three selected features comprising expression levels of miR-29a, miR-335-5p, and miR-222; wherein the at least one trained classifier classifies the epithelial non- MCC NEN according to a condition of interest as medullary thyroid carcinoma (MTC) based on elevated expression levels of miR-29a and miR-222 and reduced expression level of miR-335-5p, otherwise the at least one trained classifier classifies the epithelial NEN as non-MTC.

In one embodiment the discriminator function comprises at least one classifier trained to use at least five selected features comprising expression levels of miR-760, miR-1224-5p, miR-139, miR-205, and miR-9; wherein the at least one trained classifier classifies the epithelial non-MTC NEN according to a condition of interest as gastroenteropancreatic (GEP) neoplasm based on elevated expression levels of miR-760, miR-1224-5p, miR-139, and miR-205 and reduced expression level of miR-9, otherwise the at least one trained classifier classifies the epithelial non-MTC NEN as lung neuroendocrine neoplasm.

In one embodiment the discriminator function comprises at least one classifier trained to use at least two selected features comprising expression levels of miR-615, and miR-92b; wherein the at least one trained classifier classifies the GEP neoplasm according to a condition of interest as midgut based on elevated expression levels of miR-615 and reduced expression level of miR-92b, otherwise the at least one trained classifier classifies the GEP neoplasm as non-midgut.

In one embodiment the discriminator function comprises at least one classifier trained to use at least three selected features comprising expression levels of miR-149, miR-192, and miR-125b; wherein the at least one trained classifier classifies the midgut neoplasm according to a condition of interest as ileal neoplasm (INET) based on elevated expression level of miR-192 and reduced expression level of miR-125b and miR-149; otherwise the at least one trained classifier classifies the midgut neoplasm as appendiceal neoplasm (AppNET).

In one embodiment the discriminator function comprises at least one classifier trained to use at least two selected features comprising expression levels of miR-487b, and miR-429; wherein the at least one trained classifier classifies the midgut neoplasm according to a condition of interest as rectal neoplasm (RNET) based on elevated expression level of miR-429 and reduced expression level of miR-487b; otherwise the at least one trained classifier classifies the midgut neoplasm as pancreatic neoplasm (Pan NET).

In one embodiment the discriminator function comprises at least one classifier trained to use at least two selected features comprising expression levels of miR-18a and miR-155; wherein the at least one trained classifier classifies the lung neoplasm according to a condition of interest as carcinoid (TC/AC) based on reduced expression level of miR-18a and miR-155; otherwise the at least one trained classifier classifies the lung neoplasm as carcinoma (SCLC/LCNEC).

In one embodiment the discriminator function comprises at least one classifier trained to use at least a selected feature comprising expression level of miR-93; wherein the at least one trained classifier classifies the non-epithelial NEN according to a condition of interest as neuroblastoma (NB) based on elevated expression of miR-93; otherwise the at least one trained classifier classifies the non-epithelial NEN as non-NB.

In one embodiment the discriminator function comprises at least one classifier trained to use at least two selected features comprising expression levels of miR-10b and miR-379; wherein the at least one trained classifier classifies the non-NB neoplasm according to a condition of interest as pheochromocytoma (PCC) based on elevated expression level of miR-10b and reduced expression level of miR-379, or the at least one trained classifier classifies the non-epithelial NEN as paraganglioma (PGL) based on reduced expression level of miR-10b and elevated expression level of miR-379.

In various embodiments the data may be subjected to preprocessing prior to subjecting the data to a discriminator function.

Another aspect of the invention relates to a method for evaluating a NEN cancer in a subject, comprising: measuring expression levels of at least two miRNAs in a biological sample obtained from the subject, wherein at least a first miRNA is miR-375; determining an expression level of miR-375 relative to an expression level of at least a second miRNA in the biological sample; determining expression levels of the at least two miRNAs in biological samples obtained from subjects without cancer; wherein an elevated expression level of miR-375 relative to the expression level of the at least a second miRNA is used to determine NEN cancer status in the subject.

In one embodiment the method may comprise measuring expression levels of at least two miRNAs in first and second biological samples obtained from the subject at first and second instants in time; wherein a change in expression level of miR-375 relative to the expression level of the second miRNA across the first and second biological samples is used to determine NEN cancer status in the subject.

In one embodiment the at least a second miRNA is added to the biological sample(s). In one embodiment the at least a second miRNA is miR-159a.

In one embodiment the at least a second miRNA is at least one of miR-7, miR-451, miR-486, and miR-21.

In one embodiment the method comprises measuring expression levels using real-time quantitative polymerase chain reaction and comparing expression levels based on the delta-Ct values.

In embodiments, the biological sample(s) comprise tissue sample(s) or liquid sample(s).

In various embodiments, determining NEN cancer status may comprise one or more of differentiating between NEN and non-NEN, diagnosing a NEN pathological type, monitoring disease state and subject condition, determining prognosis, evaluating response to a treatment, evaluating effectiveness of a treatment.

Another aspect of the invention relates to a method for constructing a discriminator function including a hierarchical classifier for discriminating among a plurality of conditions of interest in a dataset. Embodiments may comprise steps of data preprocessing, feature selection and evaluation including, for example, applying techniques such as hierarchical clustering and t-distributed stochastic neighbor embedding (t-SNE); constructing individual classifiers to separate conditions of interest based on selected features and testing discriminator functions for each condition and feature; and combining all individual classifiers into a hierarchical classifier.

Another aspect of the invention relates to non-transitory computer readable media for use with a processor, the computer readable media having stored thereon instructions that direct the processor to receive data, and construct a discriminator function by: (i) subjecting the data to at least one feature selection, ranking, and evaluation algorithm, wherein at least one feature related to at least a first condition of interest is selected; (ii) using the at least one selected feature to train at least one classifier, wherein the at least one classifier outputs a first classification result for the at least a first condition of interest based on the at least one feature; and (iii) storing parameters of the at least one classifier trained for the at least a first condition of interest; wherein a discriminator function comprising the at least one classifier trained for the at least a first condition of interest is constructed.

In one embodiment the instructions direct the processor to repeat steps (i) to (iii) for a plurality of conditions of interest and store parameters of at least one classifier trained for each of the plurality of conditions of interest; wherein a discriminator function comprising a plurality of trained classifiers, wherein at least one classifier is trained for each of the plurality of conditions of interest, is constructed.

In various embodiments the data are selected from omics data, imaging data, and electromagnetic spectra data.

One embodiment comprises non-transitory computer readable media for use with a processor, the computer readable media having stored thereon instructions that direct the processor to receive data relating to expression levels of at least two selected miRNAs (miRs) in a biological sample from a subject; implement a discriminator function comprising at least one classifier trained to use at least one selected feature in the data, the at least one selected feature being related to at least a first condition of interest; and subject the data to the discriminator function; wherein the at least one trained classifier classifies a cancer according to the at least a first condition of interest based on the at least one selected feature in the data. The discriminator function may comprise a hierarchical classifier including two or more trained classifiers that classify samples in the data according to a two or more corresponding conditions of interest based on one or more selected features for each condition of interest. The two or more conditions of interest may be two or more cancer types, such as two or more NEN cancer pathologies.

Another aspect of the invention provides a method for classifying cancer in a subject, comprising: obtaining a biological sample of the cancer from the subject; measuring expression levels of at least two selected miRNAs (miRs) in the biological sample; subjecting the expression levels of the at least two miRs to a discriminator function; wherein the discriminator function classifies the cancer according to the expression levels of the at least two miRs.

In one embodiment the at least two miRs are miR-375 and miR-7; wherein the discriminator function comprises a first discriminator function that classifies the cancer as a neuroendocrine neoplasm or a non-neuroendocrine neoplasm.

In one embodiment the at least two miRs are miR-200a and miR-10b; wherein the discriminator function comprises a second discriminator function that classifies the cancer as an epithelial neuroendocrine neoplasm according to elevated expression of miR-200a and reduced expression of miR-10b, otherwise the second discriminator function classifies the cancer as a non-epithelial neuroendocrine neoplasm.

One embodiment further comprises measuring an expression level of miR-30a; subjecting the expression level of miR-30a to the discriminator function; wherein the discriminator function comprises a third discriminator function that classifies the epithelial neuroendocrine neoplasm as parathyroid adenoma (PTA) according to elevated expression of miR-30a, otherwise the third discriminator function classifies the epithelial neuroendocrine neoplasm as non-PTA.

One embodiment further comprises measuring expression levels of miR-10a and miR-212-3p; subjecting the expression levels miR-10a and miR-212-3p to the discriminator function; wherein the discriminator function comprises a fourth discriminator function that classifies the epithelial non-PTA neuroendocrine neoplasm as pituitary adenoma (PitNET) according to reduced expression of miR-10a and elevated expression of miR-212-3p, otherwise the fourth discriminator function classifies the epithelial neuroendocrine neoplasm as non-PitNET.

One embodiment further comprises measuring expression levels of miR-15b and miR-660; subjecting the expression levels of miR-15b and miR-660 to the discriminator function; wherein the discriminator function comprises a fifth discriminator function that classifies the epithelial non-PitNET neuroendocrine neoplasm as Merkel cell carcinoma (MCC) according to elevated expression of miR-15b and miR-660, otherwise the fifth discriminator function classifies the epithelial neuroendocrine neoplasm as non-MCC.

One embodiment further comprises measuring expression levels of miR-29a, miR-335-5p, and miR-222; subjecting the expression levels of miR-29a, miR-335-5p, and miR-222 to the discriminator function; wherein the discriminator function comprises a sixth discriminator function that classifies the epithelial non- MCC neuroendocrine neoplasm as medullary thyroid carcinoma (MTC) according to elevated expression levels of miR-29a and miR-222 and reduced expression level of miR-335-5p, otherwise the sixth discriminator function classifies the epithelial neuroendocrine neoplasm as non-MTC.

One embodiment further comprises measuring expression levels of miR-760, miR-1224-5p, miR-139, miR-205, and miR-9; subjecting the expression levels of miR-760, miR-1224-5p, miR-139, miR-205, and miR-9 to the discriminator function; wherein the discriminator function comprises a seventh discriminator function that classifies the epithelial non-MTC neuroendocrine neoplasm as gastroenteropancreatic (GEP) neoplasm according to elevated expression levels of miR-760, miR-1224-5p, miR-139, and miR-205 and reduced expression level of miR-9, otherwise the seventh discriminator function classifies the epithelial non-MTC neuroendocrine neoplasm as lung neuroendocrine neoplasm.

One embodiment further comprises measuring expression levels of miR-615, and miR-92b; subjecting the expression levels of miR-615, and miR-92b to the discriminator function; wherein the discriminator function comprises an eighth discriminator function that classifies the GEP neoplasm as midgut according to elevated expression levels of miR-615 and reduced expression level of miR-92b, otherwise the eighth discriminator function classifies the GEP neoplasm as non-midgut.

One embodiment further comprises measuring expression levels of miR-149, miR-192, and miR-125b; subjecting the expression levels of miR-149, miR-192, and miR-125b to the discriminator function; wherein the discriminator function comprises a ninth discriminator function that classifies the midgut neoplasm as ileal neoplasm (INET) according to elevated expression level of miR-192 and reduced expression level of miR-125b and miR-149; otherwise the ninth discriminator function classifies the midgut neoplasm as appendiceal neoplasm (AppNET).

One embodiment further comprises measuring expression levels of miR-487b, and miR-429; subjecting the expression levels of miR-487b, and miR-429 to the discriminator function; wherein the discriminator function comprises a tenth discriminator function that classifies the midgut neoplasm as rectal neoplasm (RNET) according to elevated expression level of miR-429 and reduced expression level of miR-487b; otherwise the tenth discriminator function classifies the midgut neoplasm as pancreatic neoplasm (Pan NET).

One embodiment further comprises measuring expression levels of miR-18a and miR-155; subjecting the expression levels of miR-18a and miR-155 to the discriminator function; wherein the discriminator function comprises an eleventh discriminator function that classifies the lung neoplasm as carcinoid (TC/AC) according to reduced expression level of miR-18a and miR-155; otherwise the eleventh discriminator function classifies the lung neoplasm as carcinoma (SCLC/LCNEC).

In an embodiment wherein the at least two miRs are miR-200a and miR-10b and the discriminator function classifies the cancer as a non-epithelial neuroendocrine neoplasm; the embodiment may further comprise measuring an expression level of miR-93; subjecting the expression level of miR-93 to the discriminator function; wherein the discriminator function comprises a twelfth discriminator function that classifies the non-epithelial neuroendocrine neoplasm as neuroblastoma (NB)according to elevated expression of miR-93; otherwise the twelfth discriminator function classifies the non-epithelial neuroendocrine neoplasm as non-NB.

One embodiment further comprises measuring expression levels of miR-10b and miR-379; subjecting the expression levels of miR-10b and miR-379 to the discriminator function; wherein the discriminator function comprises a thirteenth discriminator function that classifies the non-NB neoplasm as pheochromocytoma (PCC) according to elevated expression level of miR-10b and reduced expression level of miR-379, or that classifies the non-epithelial neuroendocrine neoplasm as paraganglioma (PGL) according to reduced expression level of miR-10b and elevated expression level of miR-379.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A and 1B are flowcharts showing processing steps for constructing and implementing discriminator functions according to embodiments described herein.

FIGS. 2A and 2B show miR-375 and miR-7 expression, respectively, in NEN and non-NEN samples. Normalized miR-375 and miR-7 expression was examined between 15 NEN pathological types and 7 site-matched non-NEN control groups. Site-matched NEN and non-NEN groups are designated by anatomical site: gastrointestinal-pancreatic (GEP), lung, parathyroid, skin, thyroid, and adrenal and extra-adrenal; NENs without a site-matched control were left blank (abbreviation: log2 RF, log2 normalized relative frequency).

FIGS. 3A and 3B are flowcharts showing multilayer miRNA-based classifiers for predicting NEN pathological types, according to embodiments described herein.

FIGS. 4A-4I are scatter plots showing assessment of miRNAs selected for classification, wherein epithelial and non-epithelial NENs were effectively discriminated based on miR-10b and miR-200a expression with one misclassification (FIG. 4A); within epithelial NENs, PTA, PitNET, MCC and MTC were accurately discriminated from the remaining NENs based on miR-30a expression (FIG. 4B), miR-10a and miR-212-3p expression (FIG. 4C), miR-15b and miR-660 expression (FIG. 4D), and miR-335-5p, miR-29a and miR-222 expression (FIG. 4E); lung NENs and GEP NENs were discriminated based on miR-760, miR-1224-5p, miR-139, miR-205 and miR-9 expression (FIG. 4F, FIG. 4G); within non-epithelial NENs, NB was accurately discriminated from PCC/GGL based on miR-93 expression (FIG. 4H), and PCC and PGL were separated based on miR-10b and miR-379 expression (FIG. 4I); arrows indicate misclassified samples (abbreviation: log2 RF, log2 normalized relative frequency).

FIG. 5 is a plot showing t-stochastic neighbour embedding (t-SNE) for selected classificatory miRNAs. Sample grouping was visually assessed using miRNAs selected for multilayer classification and t-SNE analysis. With one notable exception, samples clustered as epithelial or non-epithelial NENs and tended to group by pathological type. The exception was a misdiagnosed PanNET later found to be a PGL on further testing. Sample abbreviations are provided in Table 1.

### DETAILED DESCRIPTION OF EMBODIMENTS

One aspect of the invention relates to methods for classifying a neuroendocrine neoplasm (NEN) in a subject. According to certain embodiments, methods for classifying NENs may be based on comprehensive miRNA expression profiling and data mining of multiple pathological types. Reference miRNA expression profiles were generated for multiple NEN pathological types and site-matched non-NEN controls, candidate category and type specific miRNAs were identified that may be used for classification and as adjunct tissue markers, and evidence for constitutive and convergent miRNA gene expression in epithelial and nonepithelial NENs was found. Through these advances a multilayer classifier for discriminating NEN pathological types was developed, and reference profiles were provided.

In some embodiments, methods may include comparing multiple NEN pathological types and site-matched non-NEN controls using comprehensive miRNA detection through barcoded small RNA cDNA library sequencing [14] and accurate sequence annotation [10].

In some embodiments, advanced computational approaches for miRNA feature selection and classifier construction may be employed. Such approaches may include assessing data reliability through knowledge of miRNA cluster composition [5], evaluating classifier performance and transferability by determining overall and decision node level accuracy, assessing impact of miRNA cluster substitutions on accuracy, and assessing the abundance of selected classificatory miRNAs. In one embodiment, miRNA clusters may be used as a measure of data quality and transferability of miRNAs as clinical markers; and candidate miRNAs may be used to build a streamlined NEN classification tool. The identified miRNAs may also be used as mono-analyte or multi-analyte markers as needed.

In some embodiments, expression levels of at least two selected miRNAs in a biological sample are determined and are then subjected to a discriminator function that classifies cancer according to the expression levels of the at least two miRNAs. In some embodiments the biological sample may be a sample of the cancer, such as a tissue sample. In other embodiments the biological sample may be a liquid sample, such as blood.

In one embodiment at least one miRNA is miR-375. In another embodiment, at least two miRNAs are miR-375 and miR-7. Expression levels of miR-375 and miR-7 may be subjected to a discriminator function that classifies the cancer as a NEN or a non-NEN.

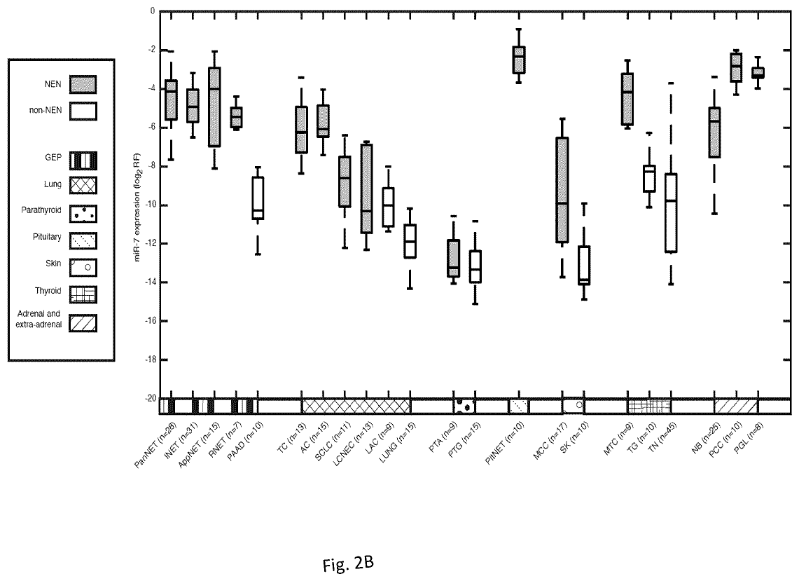

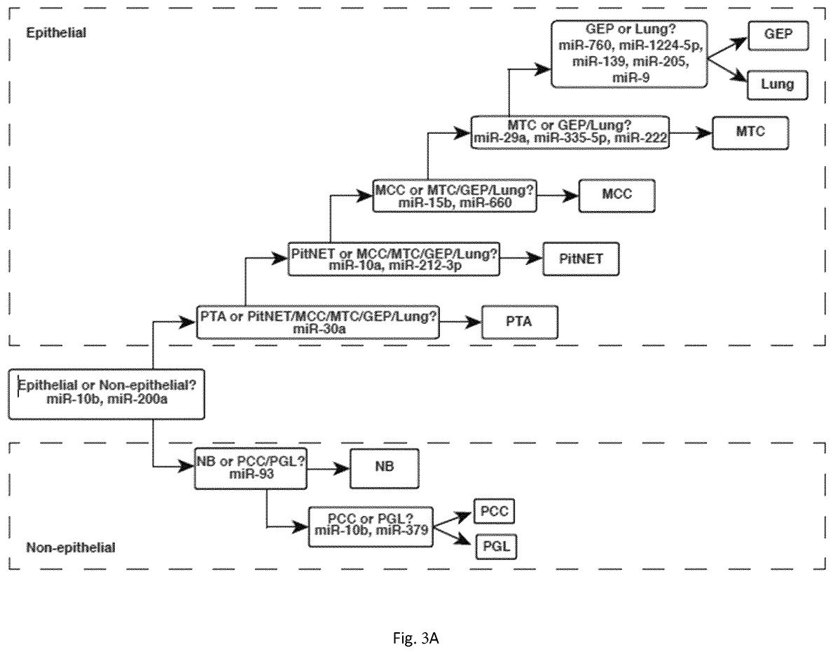

In other embodiments expression levels of additional miRNAs in the biological sample may be measured, and these expression levels additionally subjected to a discriminator function based on a hierarchical classifier having additional classifier layers. For example, after determining whether a cancer is a NEN or a non-NEN in a first classifier layer based on expression levels of at least one miR, such as miR-375, the discriminator function may then determine a NEN pathological type based on miR expression level(s) of one or more additional miR(s) in a second classifier layer. Classification may proceed this way over multiple classifier layers using one or more additional miR(s) expression level(s). For example, FIGS. 3A and 3B show embodiments of discriminator functions with multi-layer classifiers operating on expression levels of multiple miRNAs over the classifier layers. These are described in detail below. Table 3 lists and provides sequences of miRNAs that may be used in embodiments based on this approach. The sequences listed are the dominant sequences where, in general, regions comprising nucleotides 2-8 in each sequence are most important and are highly conserved. It will be appreciated that substitutions, deletions, or additions of, e.g., one or two nucleotides outside of the highly conserved regions are acceptable as such variants would not negatively affect performance of classifier embodiments.

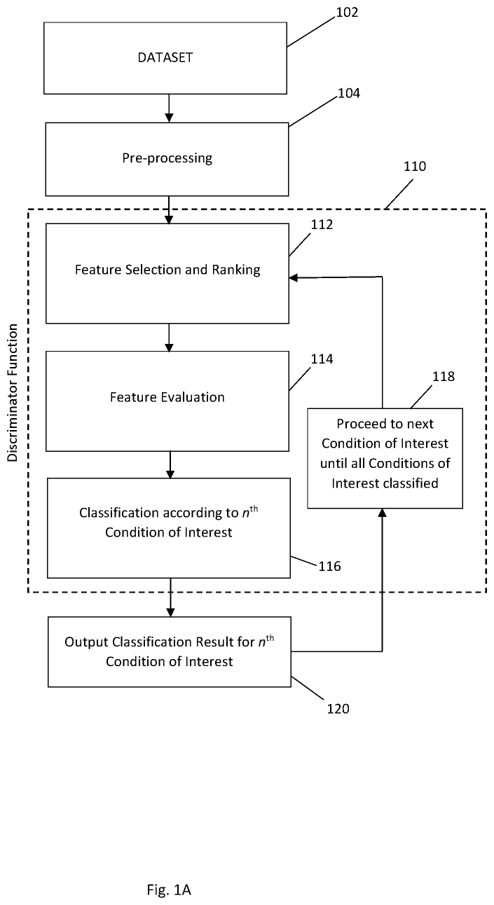

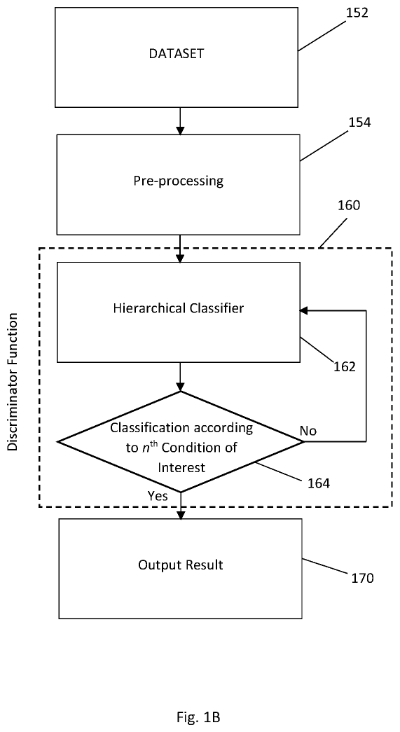

According to another aspect of the invention, methods and discriminator constructs (e.g., software products) are provided for discriminating between two or more conditions of interest (i.e., “n” conditions of interest) in a dataset. FIG. 1A is a flow chart showing a generalized process for constructing a discriminator function. Referring to FIG. 1A, a dataset (e.g., raw data) 102 obtained from a measuring, sampling, profiling, etc., regimen or modality is used as input. Examples of types of data include, but are not limited to, omics (e.g., genomics, transcriptomics, proteomics, metabolomics, etc.), imaging (e.g., ultrasound, x-ray, LiDAR, etc.) as may be applied to fields such as medical, geophysical/seismic, mechanical (e.g., non-destructive inspection), electromagnetic spectra, etc. Data are collected from samples, e.g., individual clinical specimens, biological samples, individual images, geophysical sampling locations, etc. Examples of conditions of interest include, but are not limited to: whether or not a patient has cancer or a cancer subtype, determined from omics data; whether or not a mechanical component is defective or damaged, determined from ultrasonic imaging data; whether or not an ore deposit is present, determined from seismic survey data. Whereas embodiments are described below primarily with respect to data related to miRNA expression, it will be readily understood that they are applicable to other types of data.

The data may be subjected to preprocessing 104. Preprocessing may include one or more treatment to improve overall rigor of the discriminator function. For example, preprocessing may include one or more of filtering of features (e.g., with respect to a specific threshold), normalizing, transforming, detecting and filtering sample outliers and batch effects, standardizing etc.

To construct the discriminator function several steps may be implemented at 110. First, the preprocessed data are subjected to one or more feature selection algorithms 112. A feature selection algorithm may rank data by discriminating between two class labels, i.e., between two conditions of interest. Feature selection may be performed for all pairwise comparisons of the multiple conditions of interest. The feature selection algorithm(s) may use a feature matrix and class labels as input and return a cross-validated average ranking of each feature. Since the accuracy of feature selection methods varies for different datasets, an ensemble of different feature selection algorithms based on diverse selection methods and families of predictors may be used to evaluate features according to different criteria. Cross validation may be used for all feature selection algorithms to ensure that ranking is generalizable and to avoid overfitting of the subsequent prediction model. Univariable and/or multivariable feature ranking algorithms may be used. For example, one or more feature selection algorithm may be univariable and one or more other feature selection algorithm may be multivariable. Univariable and multivariable feature ranking algorithms may rely on filter, wrapper, and/or embedded techniques. Filter techniques may include feature ranking based on one or more of, e.g., mutual information, receiver operating characteristic (ROC) criteria, Wilcoxon criteria, ReliefF analysis for classification, and Treebagger predictor importance. Wrapper feature selection algorithms may include one or more of, e.g., support vector machine (SVM), k-nearest neighbors (KNN), trees, linear discriminant analysis (LDA), logistic regression, Naïve Bayes, etc. Embedded techniques combine the characteristics of filter and wrapper methods and the feature selection may become part of a trained machine learning model, and may include Random Forests technique. The output of each feature selection may be a numeric matrix with standardized ranking, e.g., between 0 (lowest) and 1 (highest) for each feature. The results of all the algorithms may be passed through a further algorithm that performs a final ranking, such as a greedy ensemble ranking algorithm that assigns a final ranking and returns a ranked version of the input dataset. In some embodiments weighting is used to give more priority to some algorithms over others.

Following feature selection, the selected features are subjected to evaluation 114. As an example, feature evaluation may include inspecting top-ranking (e.g., the top 5% to 25%) discriminating features that can be used for building an accurate prediction model. Evaluation may include visual inspection (e.g., for grouping, wherein a higher degree of grouping of features has a greater potential for accurate classification), applying domain knowledge such as consideration of significance of a particular feature (e.g., the biological role of a protein or miR) and/or consideration of miRNA specificity between different tissues and cells, dominance within the data set (e.g., level of expression of a nucleic acid sequence or miR), supporting evidence for other miR cistron members, wherein similar expression levels of two or more cistronic miRNAs confirms data integrity (cistronic miRNAs are typically located within about 5 kb of each other in intergenic regions or within the same intron/exon and are co-transcribed and yield similar read counts for each member of a given miRNA cistron, also referred to herein as clusters). The evaluation 114 may confirm relevance of candidate features in the overall results in identification of key features in the dataset that are used as input to one or more classifiers 116. For example, in embodiments where the dataset is miRNA expression levels, evaluation results in the identification of candidate miRNAs that can be used to classify cancer. The feature selection and evaluation may result in a subset of features for building a generalizable classifier.

Following evaluation, the selected subset of features is subjected to classification 116. For example, the selected subset of features may be identified (in steps 112 and 114) as the most valuable predictors to use in constructing a classification model for all pairwise comparisons of the multiple conditions to discriminate among the multiple conditions of interest within the data. Various classification algorithms are available, for example, one or more of the classification algorithms in the MATLAB Classification Learner App (Mathworks, Inc., Natick, Mass., USA) may be evaluated during construction of the model.

Implementing the classifier model includes the discriminator function performing a hierarchical classification for each condition of the multiple (i.e., n) conditions of interest. For example, as shown in FIG. 1A, upon outputting a first classification result 120 corresponding to a first condition of interest, if there remain conditions of interest, the process is returned 118 for classification according to a next condition of interest whereupon samples are subjected to further feature selection and ranking 112, further evaluation 114, and further classification 116, and a second output includes second classification result corresponding to a second condition of interest. The hierarchical approach continues this way until all samples have been classified according to the n multiple conditions of interest. At this point a layered discriminator function is constructed with feature selection and ranking, feature evaluation, and classification functions and algorithms (i.e., parameters) that are selected and stored for each condition of then multiple conditions of interest for the particular type of data in the dataset. Consequently, the discriminator function may then be applied to another dataset of the same type of data, wherein the discriminator function automatically performs the hierarchical classification across the multiple conditions of interest. That is, no further feature selection and ranking, feature evaluation, and selection of classification algorithms is necessary to perform the hierarchical classification when the same type of data are used.

FIG. 1B is an exemplary flowchart for a process including a hierarchical discriminator function constructed as described above. The discriminator function may be constructed for use with a dataset 152 such as, for example, miRNA expression data. In a particular example related to the methods for identifying cancer described herein, the hierarchical discriminator function is constructed for classifying cancer according to multiple conditions of interest including whether the cancer is NEN or non-NEN, and further, if the cancer is NEN, then the conditions may include classifying the NEN cancer according to a specific pathological (tumor) type. As shown in FIG. 1B, the dataset is subjected to preprocessing 154. In certain embodiments, described in detail below, the constructed hierarchical discriminator function 160 may have a hierarchical classifier 162 architecture such as that shown in FIG. 3A or 3B, which show multiple conditions of interest as multiple NEN cancer subtypes. An example of a set of instructions for a hierarchical classifier such as that shown in FIG. 3B is provided in Example 2, below. According to such embodiments, hierarchical classification proceeds for the n conditions of interest 164 and the hierarchical classifier may output a classification result 170 for any of the multiple conditions of interest, e.g., NEN cancer subtypes, as shown in FIGS. 3A or 3B.

Embodiments of software products include computer code containing instructions to direct a processor to receive a dataset and implement a process for selecting features and classifiers and constructing a discriminator function as described above (e.g., a process comprising all or a part of that shown in FIG. 1A), or to direct the processor to receive a dataset and implement a discriminator function based on pre-selected features and classifiers as described above (e.g., a process comprising all or a part of that shown in FIG. 1B). Data preprocessing may of course be performed separately such that preprocessed data is input to the systems shown in FIGS. 1A and 1B at steps 112 and 162, respectively. Accordingly, data preprocessing may be an optional feature or may be omitted in software products based on FIGS. 1A and 1B. The processor may be part of a computer or a data processing system, and may execute a graphical user interface (GUI) presented on a display screen and adapted for receiving commands from a user of the system and prompting the user for input at various steps. The processor analyzes data and outputs results and displays results on the display screen. For example, the data processing system may be a server system, and the computer may be a personal computer (PC) or tablet-based computer.

Another aspect of the invention provides programmed media for use with a processor, comprising code stored on non-transitory storage media compatible with the processor, the code containing instructions to direct the processor to receive a dataset and implement a process for constructing a discriminator function as described above (e.g., a process such as that shown in FIG. 1A), or to direct the processor to receive a dataset and implement a discriminator function as described above (e.g., a process such as that shown in FIG. 1B).

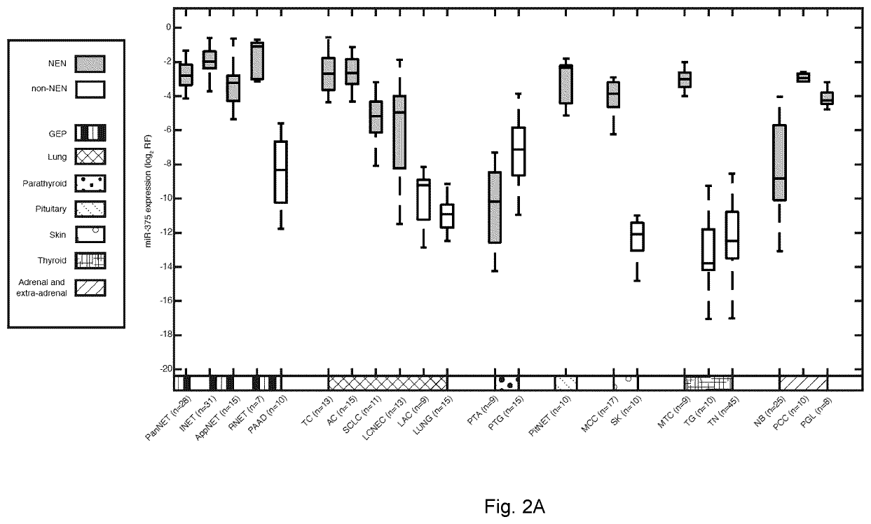

According to another aspect of the invention, there are provided herein comprehensive data (e.g., Tables 1A and 1B, FIGS. 2A and 2B) showing for the first time elevated expression of miR-375 in a broad range of NEN pathological types, thus demonstrating the utility of miR-375 as a pan-NEN biomarker. As described herein, the expression level of miRNA may be measured in substantially any NEN pathological type to provide a rapid and convenient assessment of a patient. Practical uses may include, for example, differentiating between NEN and non-NEN, diagnosing a NEN pathological type, monitoring disease state/patient condition, determining prognosis, evaluating response to a treatment, evaluating effectiveness of a treatment, etc.

Thus, provided herein are methods for one or more of diagnosing a NEN pathological type, monitoring disease state/patient condition, determining prognosis, evaluating response to a treatment, and/or evaluating effectiveness of a treatment in a subject with a NEN cancer. The methods comprise obtaining a biological sample from the subject and measuring expression levels of at least two miRNAs in the biological sample, wherein at least a first miRNA is a biomarker in NEN cancer. In some embodiments, the first miRNA is miR-375 and at least a second miRNA may be one or more other miRNAs that may be present in the biological sample. Examples of other miRNAs that may be present in the biological sample include, but are not limited to, miR-7, miR-21, miR-451, and miR-486. In some embodiments, the first miRNA is miR-375and at least a second miRNA is added to the biological sample as a standard. An example of a miRNA that may be added to the sample as a standard is a miRNA that may be easily detected, e.g., a non-human miRNA, such as miR-159a. The expression levels of the at least a first miRNA and the at least a second miRNA are compared, wherein a high expression level of the at least a first miRNA relative to the at least a second miRNA is indicative of NEN in the subject. Comparing may include comparing miRNA expression levels to control levels, such as levels of the miRNAs in samples from subjects without cancer. Comparing may include comparing the expression level of the at least a first miRNA to the expression level of the standard added to the biological sample. Moreover, by obtaining two or more measures of the at least a first miRNA from the subject over time, prognosis, response to a treatment, and/or effectiveness of a treatment in the subject with a NEN cancer can be determined.

In one embodiment the miRNA expression levels may be measured using real-time quantitative polymerase chain reaction and an expression level of the at least a first miRNA s may be generated based on comparison to that of at least a second miRNA using the delta-Ct method. In one embodiment the biological sample may be a sample of the cancer, such as a tissue sample. In other embodiments the biological sample may be a liquid sample, such as blood. In one embodiment the at least a first miRNA is miR-375.

### ABBREVIATIONS

The following abbreviations are used throughout this description:
AC—atypical carcinoid
AE1/AE3—anti-cytokeratin antibodies
AppNET—appendiceal NET
CgA—chromogranin A
FFPE—formalin-fixed paraffin-embedded
FF—fresh-frozen
GATA3—“GATA”-binding transcription factor 3
GEP—gastrointestinal-pancreatic
INET—intestinal NET
K-W—Kruskal-Wallis
LAC—lung adenocarcinoma
LCNEC—large cell neuroendocrine carcinoma
LUNG—non-diseased lung
MCC—merkel cell carcinoma
miRNA, miR—microRNA
mRNA—messenger RNA
MTC—medullary thyroid carcinoma
NB—neuroblastoma
NEC—neuroendocrine carcinoma
NEN—neuroendocrine neoplasm
NET—neuroendocrine tumor
PAAD—pancreatic adenocarcinoma
PanNET—pancreatic NET
PCC—pheochromocytoma
PGL—paraganglioma
PitNET—pituitary NET
PTA—parathyroid adenoma
PTG—parathyroid gland
RNET—rectal NET
SCLC—small cell lung carcinoma
SK—skin
SVM—support vector machine
SYP—synaptophysin
TC—typical carcinoid
TG—thyroid gland
TN—thyroid neoplasm
t-SNE—t-stochastic neighbour embedding

Embodiments are further described by way of the following non-limiting Examples.

### EXAMPLE 1

Described below are methods and materials used for miRNA expression profiling, data mining and machine learning, and the development of a NEN classification tool for multiple pathological types.

### Study Design and Clinical Materials

Sequencing-based miRNA expression profiles from 378 clinical samples, comprising 239 NEN cases and 139 site-matched non-NEN controls, were used. Expression profiles were either compiled from published studies [4,6-9] (n=149) or generated through small RNA sequencing (n=229). The use of de-identified clinical data and banked or archived clinical materials was approved through the Research Ethics Board at Queen's University, the Institutional Review Boards of Memorial Sloan Kettering Cancer Center, The Rockefeller University and Weill Cornell Medicine, and the Medical Ethics Committee at the Amsterdam University Medical Center.

### RNA Isolation and Quantitation

Total RNA was isolated from 306 formalin-fixed paraffin-embedded tissue blocks and 72 fresh-frozen tissue samples using the Qiagen RNeasy® Mini Kit (n=258), TRIzol™ Reagent (n=68), the Ambion RecoverAll™ Total Nucleic Acid Isolation Kit (n=28), Amsbio RNA-Bee™ Isolation Reagent (n=10) and Qiagen miRNeasy® Mini Kit (n=5), according to the manufacturers' instructions or as described [4,6-9]. Total RNA concentrations were measured using the Qubit™ fluorometer (n=258), NanoDrop® ND-1000 spectrophotometer (n=61) or Agilent 2100 Bio-analyzer (n=28). RNA isolation and quantitation data were unavailable for 9 (2.4%) and 31 (8.2%) samples, respectively.

### Small RNA Sequencing and Sequence Annotation

miRNA expression profiles for all 378 samples were generated using an established small RNA sequencing approach and sequence annotation pipeline [5]; spiked-in oligoribonucleotide calibrator markers enabled miRNA quantitation in each sample. Small RNA cDNA libraries were sequenced on HiSeq 2500 Illumina platforms in the Genomics Resource Center, The Rockefeller University, the McGill University and Génome Québec Innovation Center, and the Genomics Core, Albert Einstein College of Medicine. FASTQ sequence files were annotated through an automated pipeline (rnaworld.rockefeller.edu) [5], yielding sequencing statistics and merged miRNA, miRNA cluster and calibrator read counts. Merged miRNA refers to combined counts of multicopy miRNAs from different genomic locations and miRNA clusters are transcriptional units as defined [10]; the term “miRNA” hereafter refers to merged miRNA data. miRNA content was calculated using total RNA and calibrator RNA input ratio multiplied by total miRNA and calibrator count ratio as described [4].

### Data Preprocessing and Filtering

Data preprocessing, filtering and subsequent analyses were performed in MATLAB (Mathworks, Inc., Natick, Mass., USA, version R2019a as described [9]). To report miRNA abundance independent of sequencing depth, read counts were normalized against total sequence reads annotated as miRNAs. Sample outliers and batch effects were identified through correlation analyses [11] of miRNA expression profiles and excluded from the final dataset to increase study rigor. These analyses were completed for each NEN pathological type or site-matched non-NEN control group prior to preprocessing of the combined sample set. Sequencing data were of sufficient quality for 221 (92%) of 239 NEN cases and 114 (82%) of 139 non-NEN controls. Most excluded samples were individual outliers, except for 10 non-NEN samples from a single sequencing run. Following preprocessing, all non-human miRNAs and human miRNA STAR sequences were excluded from further analyses. To exclude miRNAs or miRNA clusters with low expression across samples, a filtering threshold was applied as described [3]; specific filtering thresholds were set as a percentile of overall expression as indicated below.

Unsupervised Hierarchical Clustering of Filtered miRNA Expression Profiles

To assess sample grouping, unsupervised hierarchical clustering was performed using log2 transformed normalized read counts of miRNA and miRNA clusters from all preprocessed samples. Euclidean distance was used as the similarity parameter with complete agglomeration hierarchical clustering applied in the heatmap.2 function of the R gplots package (www.rdocumentation.org/packages/gplots/versions/3.0.1.1). Lowly expressed miRNAs and miRNA clusters were excluded with the filtering threshold set as the top 75% abundant miRNA and clusters in at least one sample.

Assessment and Comparative Analyses of Abundant miRNAs in NEN and Non-NEN Samples

To identify candidate miRNA markers for all NENs and for each NEN pathological type, miRNAs and miRNA clusters were ranked by abundance and the top 1% were considered. These abundant miRNAs and miRNA clusters were compared and correlated between NEN cases and non-NEN controls, as well as between each pathological type and site-matched non-NEN control group. To highlight substantial differences in miRNA expression, only comparisons with 20-fold or greater difference are discussed. For single-member miRNA clusters, abundance measures approximate the abundance of the single miRNA, and are not separately discussed.

Discovery Analyses for miRNA-Based NEN Classification

To identify miRNAs or miRNA clusters that accurately discriminate between or within epithelial or non-epithelial NENs, an established feature selection algorithm [11] was used. The algorithm is an ensemble of 13 different machine learning techniques with 5-fold cross-validation. To prioritize high expression, the filtering threshold was set to the 90th percentile; miRNAs or miRNA clusters expressed above this threshold in >5% of samples were retained. Next, miRNAs and miRNA clusters that discriminate epithelial from non-epithelial NENs were compared and ranked. Subsequently, miRNA markers that successively identified epithelial NENs were compared and ranked, including parathyroid adenoma (PTA), pituitary adenoma (PitNET), Merkel cell carcinoma (MCC), medullary thyroid carcinoma (MTC) and lung NENs from gastrointestinal-pancreatic (GEP) NENs, respectively. Lastly, miRNA markers were identified that discriminated neuroblastoma (NB), pheochromocytoma (PCC) and extra-adrenal paraganglioma (PGL) from each other within the non-epithelial group were compared and ranked. Only the top-ranking 3% miRNAs and miRNA clusters in these comparisons were assessed for classification below.

### Construction and Cross-Validation of Multilayer Classifier

Scaling our existing approach to miRNA-based NEN classification [9,11], a multilayer classifier for discriminating NEN pathological types based on selected miRNAs was constructed and cross-validated. For each decision layer, all available algorithms (n=23) in the MATLAB Classification Learner App were evaluated using 5-fold cross-validation. In each case, the classification model with highest accuracy, e.g., a support vector machine (SVM) classifier, was used to identify the smallest subset of miRNAs with the most discriminatory power for comparisons as above. Based on these subsets, a multilayer classifier was constructed in which miRNA expression profiles were subjected to a discriminator function at each layer, through which they were first classified as epithelial or non-epithelial prior to classification to a specific pathological type at subsequent layers (e.g., see FIGS. 3A and 3B).

### Assessment of Classifier Performance and Transferability

To assess the performance and transferability of our multilayer classifier, t-stochastic neighbor embedding (t-SNE) was used to visualize sample grouping patterns based on miRNAs selected for classification. Overall classifier accuracy was determined, the impact of miRNA cluster member substitutions on classifier accuracy was evaluated, and the expression levels of the selected miRNAs was inspected.

### Statistical Analyses

Statistical analyses of clinical parameters were performed using SPSS Statistics (IBM, Armonk, N.Y., USA, version 25) and MATLAB. Differences in miRNA content and normalized miRNA expression were evaluated between NEN and non-NEN samples, and within NEN pathological types using the non-parametric Kruskal-Wallis (K-W) test; a two-sided P-value of <0.05 was considered significant. Similarities in miRNA expression between samples were determined using Spearman's correlation.

### RESULTS

### Anatomical Distribution and Histopathological Diagnoses of Study Samples

To characterize and compare miRNA expression between NEN and non-NEN samples, relevant study materials were collected, comprehensive miRNA expression profiles were generated through barcoded small RNA sequencing, and quality controlled profiles were generated through data preprocessing and downstream analyses using statistical and machine learning approaches. Following data preprocessing for quality control, the final study cohort included 221 NEN cases and 114 site-matched non-NEN controls, hereafter termed study samples (Table 1). NEN cases included 15 distinct pathological types, arising in seven anatomical sites, including the gastrointestinal tract and pancreas, lung, parathyroid gland, pituitary gland, skin, thyroid gland, and the adrenal gland and extra-adrenal sites. Site-matched non-NEN controls included non-diseased tissues and non-NEN cancers from five anatomical sites, including the pancreas, lung, parathyroid gland, skin and thyroid gland.

Small RNA Sequencing of Study Samples Comprehensive miRNA expression profiles were generated for all samples through barcoded small RNA sequencing.

Following sequence annotation, a median of 4,386,727 (range: 53,516-40,305,4453) total small RNA reads and 258,932 (range: 1,312-3,723,507) calibrator reads were obtained. For miRNAs, a median of 2,322,722 (range: 1,740-34,781,174) miRNA sequence reads were detected, representing a median of 63.1% total sequence reads; miRNA, miRNA cluster and calibrator expression profiles for each sample were subsequently generated from these reads. Median miRNA content was 26.4 (range: 0.0-2048.4) fmol/μg total RNA.

Abundant miRNA Composition in NEN and Non-NEN Samples

To better understand miRNA composition in NEN and non-NEN samples, abundant miRNAs and miRNA cistrons within and between sample sets were assessed and correlated. Abundant miRNA and miRNA cluster composition was similar within all NEN cases or all non-NEN controls. The number of members in each miRNA cluster is indicated in parentheses following the cluster name, e.g., cluster-hsa-miR-98(13). Among all NEN cases, miR-375, -21, -143, -let-7a, -26a, -7, -let-7f and -125b and cluster-miR-375(1), -98(13), -21(1) and -23a(6) were the most abundant miRNAs and miRNA clusters, with the median relative frequency ranging 1.5-10.6% and 3.6-10.6% of respective total read counts. Within this group, miR-375, -21, -143, -let-7a, -26a, -7, -let-7f, -125b and -141 and cluster-miR-98(13), -miR-375(1), -miR-7-1(3) and -miR-143(2) were highly expressed in five or more pathological types. In comparison, among all non-NEN controls, miR-21, -125b, -let-7a, -143, -let-7f, -30a, -26a and -29a and cluster-miR-98(13), -21(1), -30a(4) and -23a(6) were the most abundant miRNA and miRNA clusters, ranging 2.5-10% and 5.2-15.9% of respective total miRNA-annotated read counts. Within this group, miR-21, -let-7a, -143, -30a, -let-7b and -30d and cluster-miR-98(13), -miR-21(1), -miR-23a(6) and -miR-30a(4) were highly expressed in five or more non-NEN entities. Correlation analyses highlighted the similarities in abundant miRNA composition within epithelial and non-epithelial NENs; with the exception of PTA, NEN cases were less correlated with site-matched non-NEN controls.

Comparative Analyses of Abundant miRNAs in NEN and Non-NEN Samples

To better understand meaningful differences in miRNA composition between NEN and non-NEN samples, abundant miRNAs and miRNA were compared for all NEN samples and for each pathological type with relevant controls. Comparative analyses indicated that miR-375 and miR-7 were 216- and 48-fold higher in all NEN cases compared to all non-NEN controls, respectively. Fold changes ranged 59-816- and 41-69-fold higher for miR-375 and miR-7 in specific NEN pathological types compared to site-matched non-NEN controls (FIGS. 2A and 2B, respectively). The only exception was observed in PTA, which showed the lowest miR-375 and miR-7 expression of all NENs; in fact, higher expression was observed in non-neoplastic parathyroid glands. Other notable miRNA over-expression among NENs included miR-127, with 86-fold higher expression in typical carcinoids (TC) compared to lung non-NEN tissues; cluster-miR-127(8) was also 78-fold higher in TC compared to lung non-NEN tissues (data not shown). In addition, miR-203 and miR-205 expression was 143-and 366-fold higher in non-NEN skin controls than MCC, respectively.

Unsupervised Hierarchical Clustering of Filtered miRNA Expression Profiles

To assess the classificatory potential of miRNA expression profiling, the data were explored with unsupervised hierarchical clustering using Euclidean distance and complete agglomeration hierarchical clustering was performed using filtered (union of top 75% abundance) log2 normalized miRNA sequence reads for all NEN cases (n=221) and non-NEN controls (n=114). With the exception of all PTA samples and one large-cell NEC (LCNEC) sample, NEN cases and non-NEN controls clustered separately. In addition, epithelial samples clustered distinctly from non-epithelial samples with the exception of one pancreatic NET (PanNET). NEN pathological types preferentially clustered together rather than with site-matched non-NEN controls. Unsupervised hierarchical clustering of filtered miRNA cluster expression from the same samples clustered as above.

Discovery Analyses for miRNA-Based NEN Classification

Next, candidate miRNA markers were identified for NEN classification using an established approach comprising feature selection and validation [9]. Using this approach, effective miRNA markers were selected from the top-ranked 3% miRNAs or miRNA clusters discriminating between or within epithelial or non-epithelial NENs. These comparisons were used to construct and assess the reliability of the multilayer classifier below. Sequences of miRNAs used for classification as NEN or non-NENE, and for NEN classification, are provided in Table 3.

### Construction and Cross-Validation of Multilayer Classifier

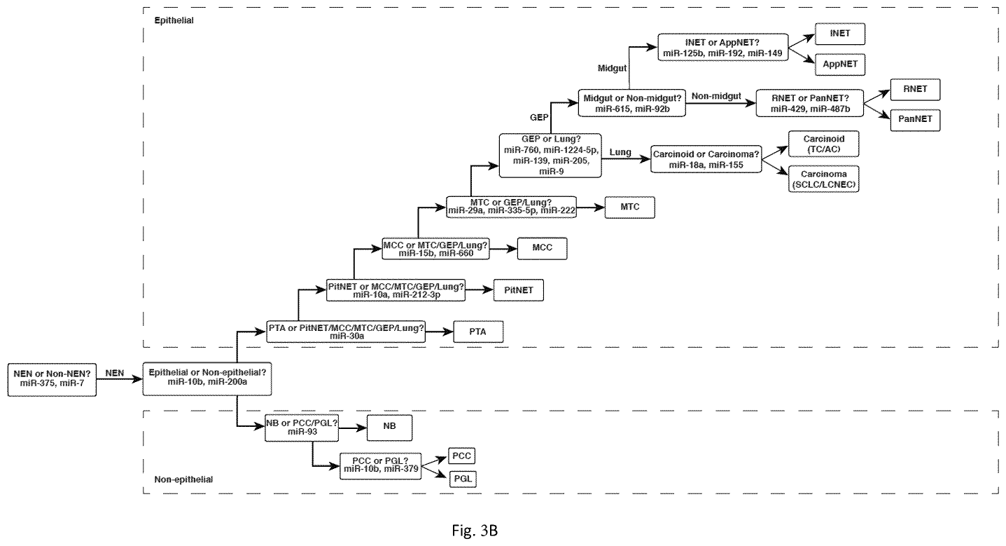

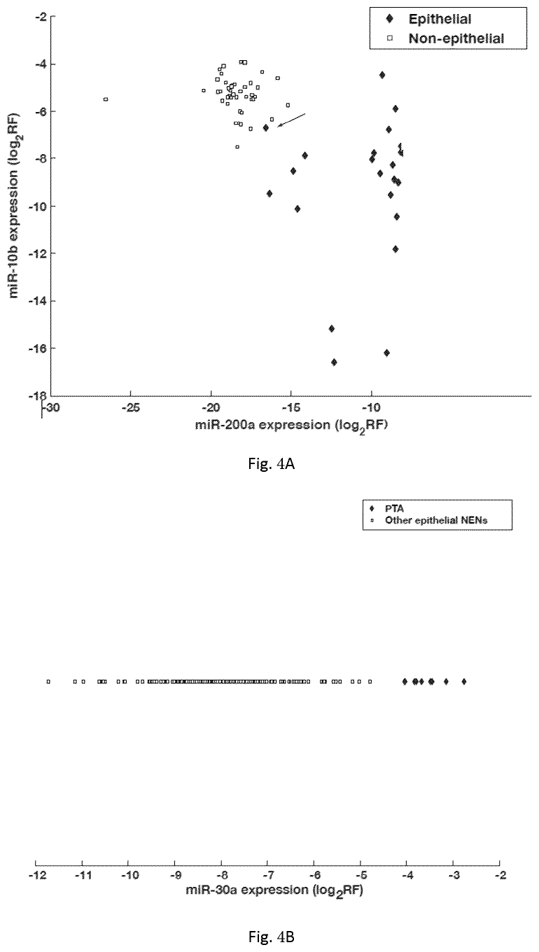

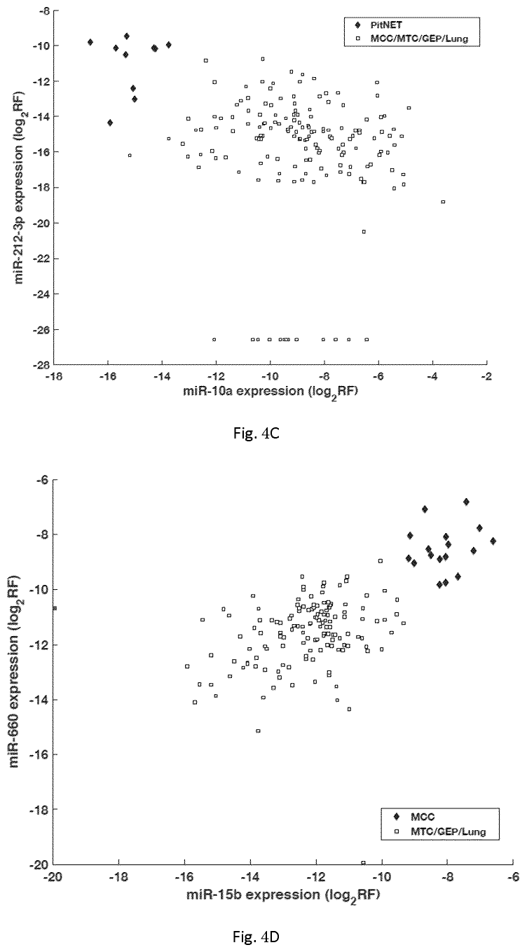

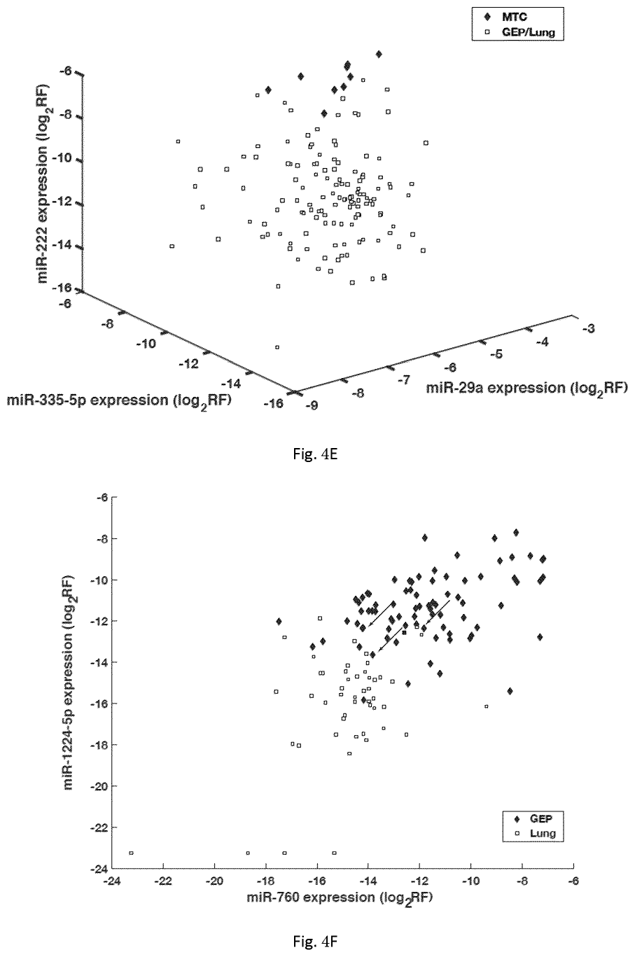

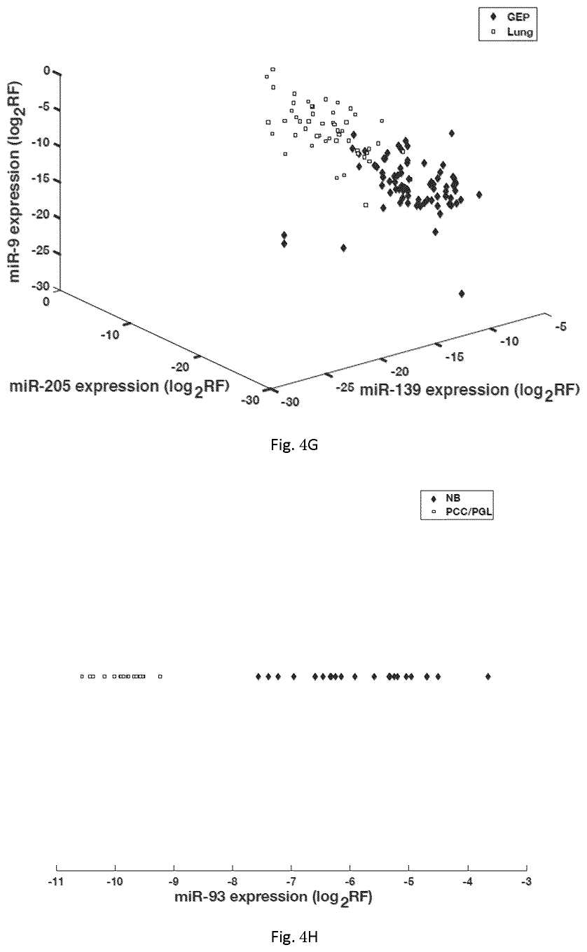

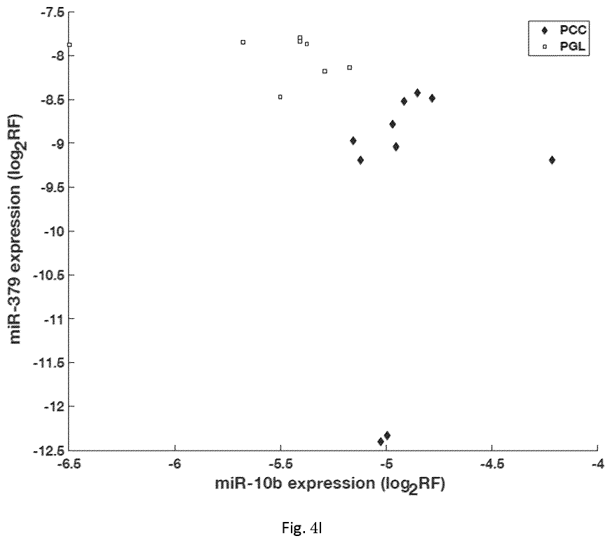

Multilayer miRNA-based classifiers for predicting NEN pathological types with 5-fold cross-validation were constructed and assessed for accuracy. One embodiment with multiple layers is shown in FIG. 3B. In another embodiment, shown in FIG. 3A, the classifier had eight decision layers. One embodiment may use a linear or cubic SVM model at each layer. In the first layer, miR-200a expression was significantly higher in epithelial than non-epithelial NENs (K-W P-value <0.01); miR-10b provided additional prediction power (K-W P-value <0.01). When combined, these two miRNAs discriminated epithelial and non-epithelial NENs with only one sample misclassification (FIG. 4A), which was found to be a histological misidentification (see below). In subsequent layers, sample profiles were successively assigned to other pathological types using the least number of miRNAs required. Within epithelial NENs, PTA, PitNET, MCC and MTC were, respectively, discriminated from remaining NENs based on expression of miR-30a, miR-10a and miR-212-3p, miR-15b and miR-660, and miR-335-5p, miR-29a and miR-222 (FIGS. 4B-4E). Lung NENs and GEP NENs were discriminated based on expression of miR-760, miR-1224-5p, miR-139, miR-205 and miR-9 with three misclassifications (FIGS. 4F and 4G). Within non-epithelial NENs, NB and PCC/PGL, and PCC or PGL, were accurately discriminated based on expression of miR-93, and miR-10b and miR-397, respectively (FIGS. 4H and 4I ). Decision node level accuracy ranged from 97% to 100%. Similar results were generated using relevant miRNA cluster data and are not presented.

### Assessment of Classifier Performance and Transferability

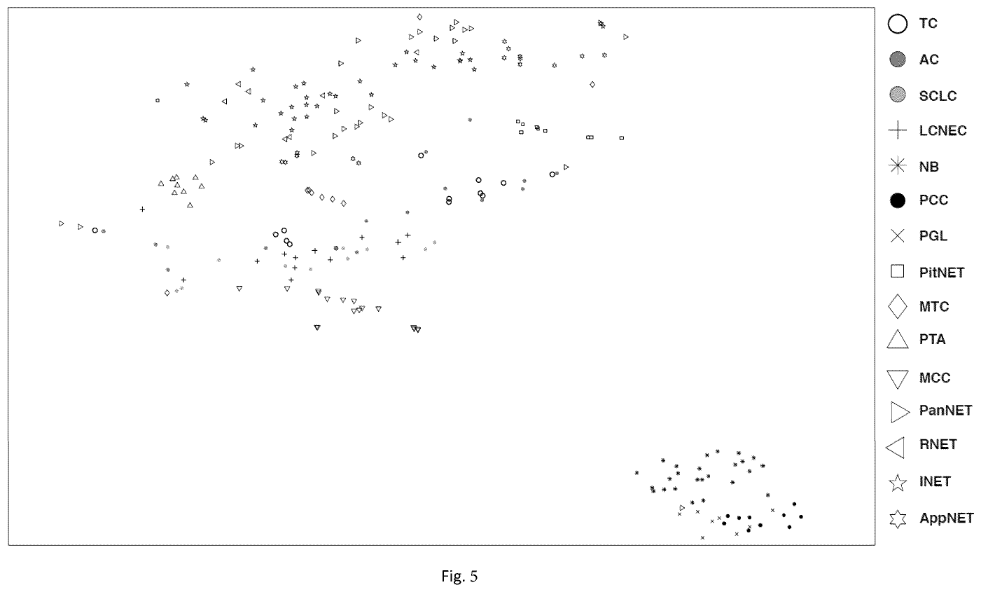

Using the 17 miRNAs selected for multilayer classification in the embodiment of FIG. 3A, t-SNE analysis indicated clear separation of epithelial and non-epithelial NENs with one notable exception (FIG. 5), which was found to be a histological misidentification (see below). NEN pathological types also grouped together within epithelial and non-epithelial clusters. With 217 of 221 samples accurately classified, the overall accuracy of the multilayer classifier was 98% (Table 2). miRNA cluster substitutions had little to no effect on overall and decision node level accuracy (data not presented). At each decision node of the classifier, selected miRNAs were always more highly expressed in one comparison group (0.40%; range: 0.01-7.82%) versus the other (0.03%; range: 0.00-2.35%), highlighting their utility as translatable tissue markers of specific NEN pathological types.

Detection of Histological Misidentification by miRNA-Based NEN Classifier

The unusual finding of an epithelial PanNET within the cluster of non-epithelial NENs (FIGS. 4A and 5), in addition to miRNA-based classification of this PanNET as a PGL (Table 2), prompted a review the histopathology of this case. Upon review, the tumor was a small (<1 cm in size) low-grade NET at the tail of the pancreas, with histological features overlapping both PanNET and PGL. Immunohistochemical analysis showed that the tumor cells were diffusely positive for synaptophysin, chromogranin A and GATA3, and negative for cytokeratin (AE1/AE3 antibodies). This phenotype diagnosed this tumor as a PGL, as predicted by the miRNA classifier, and not a PanNET, which should be cytokeratin-positive and GATA3-negative [12,13]. The unusual case was misidentified based on initial histology, but was correctly diagnosed by molecular profiling and miRNA-based classification.

### DISCUSSION

Using unsupervised hierarchical clustering of filtered miRNA expression profiles provided new knowledge of NEN grouping. With the exception of all PTA and one LCNEC sample, NEN cases and non-NEN controls clustered separately. Based on these findings, it is suggested that all PTA have a distinct gene expression pattern linked to their indolent behavior; the LCNEC sample showed areas of possible squamous cell differentiation (data not shown) that may explain peculiar clustering patterns. Within NENs, two major groups corresponding to epithelial and non-epithelial NENs are evident; interestingly, one epithelial NEN clusters with non-epithelial NEN samples. The results show that these epithelial and non-epithelial NENs can be discriminated through miR-200a and miR-10b expression, and confirm that the epithelial PanNET sample is actually a non-epithelial PGL based on additional cytokeratin and GATA-3 immunostaining. Within non-NENs, samples group mostly by anatomical site of origin as expected [3]. Visual inspection of cluster diagrams indicates similarities and differences in abundant miRNA composition in NEN and non-NEN samples.

Similarities in abundant miRNA composition between samples provide coarse insights into cellular gene expression programs. Within NENs, miR-375, -21, -143, -let-7a, -26a, -7, -let-7f, -125b and -141 were highly expressed in five or more pathological types. miR-375, the most abundant miRNA in NENs, is believed to regulate lineage-specific differentiation, growth, and function of neuroendocrine cells. Correlation analyses highlighted similarities in abundant miRNA composition for all NENs, including epithelial or non-epithelial NENs. These findings indicate that all NENs have a constitutive miRNA gene expression program that likely directly or indirectly maintains the neuroendocrine cell phenotype. Given the different cellular ori gins of epithelial and non-epithelial NENs, convergent miRNA gene expression likely implies functional similarities. Within non-NEN samples, miR-21, -let-7a, -143, -30a, -let-7b and -30d were highly expressed in five or more non-NEN entities.

Differences in abundant miRNA composition between samples can also be used to identify new and confirm known miRNA markers. miR-375 expression was substantially higher in all NEN cases compared to non-NEN controls. Where comparisons allowed, miR-375 was consistently higher in NEN pathological types compared to site-matched non-NEN controls. Based on current miRNA expression tissue atlases, miR-375 is currently believed to be an endocrine gland specific marker [3,15]. However, the presence of miR-375 in enteroendocrine cells, pancreatic beta cells, thyroid C cells, and MCC, NB, and SCLC cell lines suggests that miR-375 is a neuroendocrine cell marker. Given the specificity and distribution of miR-375 in the samples herein and its reported abundance in seemingly disparate NEN pathological types, it is suggested herein that miR-375 is a universal marker of neuroendocrine cell differentiation. miR-375 appears to be highly expressed in NENs, in amounts proportional to the number of normal neuroendocrine cells and/or the degree of neuroendocrine differentiation within control tissues or tumors; neuroendocrine differentiation of tumors is more common than currently appreciated [16]. More systematic studies will confirm this suggestion.

Although less abundant than miR-375, miR-7 expression was also elevated in all NEN cases compared to non-NEN controls. Where comparisons allowed, miR-7 was often higher in NEN pathological types compared to site-matched non-NEN controls. Other than expression in the pituitary gland, atlas studies provide limited information on miR-7 expression [3,15]. However, the presence of miR-7 in enteroendocrine cells, pancreatic islet cells, thyroid C cells, but not controls suggests that this miRNA also has some degree of neuroendocrine specificity. Thus, provided herein is a method for determining NEN or non-NEN based on miR-375 and miR-7 expression, wherein high miR-375 and miR-7 indicate NEN, otherwise, low miR-375 and miR-7 indicate non-NEN. The determination of NEN or non-NEN may be made using differential expression techniques or using a classifier, for example as shown in Example 2. Given their specificity, some prior tissue profiling studies may have inadvertently interpreted miR-375 or miR-7 reduction in expansile cancer lesions as miRNA reduction rather than neuroendocrine cell destruction. Although miR-127 was higher in TC than non-NEN controls, the significance of this difference is unclear. Conversely, comparisons of abundant miRNA composition between non-NEN and NEN samples identified known tissue-specific miRNA markers such as miR-203 and miR-205 [3].

A feature selection algorithm was used to identify 17 miRNAs to discriminate 15 NEN pathological types (FIG. 3A); t-SNE analyses using these miRNAs clearly separated epithelial and non-epithelial NENs and provided clustering by pathological type. Given their classificatory potential, these miRNAs were used to construct and validate multilayer classifiers for discriminating NEN pathological types. One embodiment correctly identified 217 (98%) of 221 samples. Three of the four misclassified samples occurred at the GEP NEN versus lung NEN decision node, suggesting model over-fitting and the need for additional samples for validation. On further testing, the fourth ‘misclassified’ sample turned out to be a PGL as indicated by miRNA expression profiling. Criteria for evaluating classifier performance and transferability, including determining overall and decision node level accuracy, assessing the impact of miRNA cluster substitutions on classifier accuracy, and showing the relative abundance of miRNAs selected for classification were developed.

TABLE 1
Anatomical distribution and histopathological
diagnoses of study samples.

| | Number of samples, n | | Number of samples, n |
|---|---|---|---|
| NENS | (%) | non-NENs | (%) |
| Total | 221 | | 114 |
| Epithelial | | | |

Gastrointestinal tract and pancreas

| | | | | | |
|---|---|---|---|---|---|
| PanNET | 28 | (13%) | PAAD | 10 | (9%) |
| INET | 31 | (14%) | | | |
| AppNET | 15 | (7%) | | | |
| RNET | 7 | (3%) | | | |

Lung

| | | | | | |
|---|---|---|---|---|---|
| TC | 13 | (6%) | LAC | 9 | (8%)^{a} |
| AC | 15 | (7%) | LUNG | 15 | (12%)^{a} |
| SCLC | 11 | (5%) | | | |
| LCNEC | 13 | (6%) | | | |

Parathyroid gland

| | | | | | |
|---|---|---|---|---|---|
| PTA | 9 | (4%) | PTG | 15 | (13%) |

Pituitary gland

| | | |
|---|---|---|
| PitNET | 10 | (5%) |

Skin

| | | | | | |
|---|---|---|---|---|---|
| MCC | 17 | (8%) | SK | 10 | (9%) |

Thyroid

| | | | | | |
|---|---|---|---|---|---|
| MTC | 9 | (4%) | TG | 10 | (9%)^{b} |

| | | |
|---|---|---|
| TN | 45 | (39%)^{b} |

Non-epithelial
Adrenal gland and extra-adrenal sites

| | | |
|---|---|---|
| NB | 25 | (11%) |
| PCC | 10 | (5%) |
| PGL | 8 | (4%) |

^{a}For lung NENs, neoplastic (LAC) and non-diseased (LUNG) tissue controls were available.
^{b}For MTC, neoplastic (TN) and non-diseased (TG) tissue controls were available.

Anatomical location and diagnostic histopathological information are presented for 221 NEN cases, comprising 15 pathological types from seven anatomical sites, and 114 site-matched non-NEN controls, comprising seven diagnostic entities from five anatomical sites. Sample abbreviations: AC, atypical carcinoid; AppNET, appendiceal NET; INET, ileal NET; LCNEC, large-cell NEC; MCC, Merkel cell carcinoma; MTC, medullary thyroid carcinoma; NB, neuroblastoma; PanNET, pancreatic NET; PCC, pheochromocytoma; PGL, paraganglioma; PitNET, pituitary adenoma; PTA, parathyroid adenoma; RNET, rectal NET; SCLC, small-cell lung carcinoma; TC, typical carcinoid. Non-NEN samples comprise lung (LUNG), lung adenocarcinoma (LAC), pancreatic adenocarcinoma (PAAD), parathyroid gland (PTG), skin (SK), thyroid gland (TG) and thyroid neoplasm (TN).

TABLE 2
Overall accuracy of multilayer classifier for discriminating NENs.
Established Diagnosis

| | | GEP | Lung | | | | | | | |
|---|---|---|---|---|---|---|---|---|---|---|
| | | NET | NET | MTC | MCC | PitNET | PTA | PCC | PGL | NB |
| Multilayer | GEP | 80 | 3 | | | | | | | |
| classifier | NET | | | | | | | | | |
| designation | Lung | | 49 | | | | | | | |
| | NET | | | | | | | | | |
| | MTC | | | 9 | | | | | | |
| | MCC | | | | 17 | | | | | |
| | PitNET | | | | | 10 | | | | |
| | PTA | | | | | | 9 | | | |
| | PCC | | | | | | | 10 | | |
| | PGL | 1 | | | | | | | 8 | |
| | NB | | | | | | | | | 25 |
| Decision-level | | 80/81 | 49/52 | 9/9 | 17/17 | 10/10 | 9/9 | 10/10 | 8/8 | 25/25 |
| accuracy | | (99%) | (94%) | (100%) | (100%) | (100%) | (100%) | (100%) | (100%) | (100%) |

| | |
|---|---|
| Overall | 217/221 |
| accuracy | (98%) |

Using the multilayer classifier of FIG. 3A, NEN miRNA profiles were assigned to one of nine pathological subgroups or pathological types. Cases of GEP NENs (AppNET, INET, PNET, RNET) or lung NENs (TC, AC, SCLC, LCNEC) were not assigned to individual pathological types because of previously developed miRNA-based classifiers for these subgroups [9]. By comparing classifier designations to established histopathological diagnoses, overall classifier accuracy was 98%. Additional measures of classifier performance were also calculated: precision (0.98), recall (0.99) and Matthews correlation coefficient (0.98). Sample abbreviations are provided in Table 1.

TABLE 3
Sequences of human microRNA markers used in
embodiments.

| miRNA | miRNA sequence | SEQ ID NO. |
|---|---|---|
| miR-375 | UUUGUUCGUUCGGCUCGCGUGA | 1 |
| miR-7 | UGGAAGACUAGUGAUUUUGUUGUU | 2 |
| miR-10b | UACCCUGUAGAACCGAAUUUGU | 3 |
| miR-200a | UAACACUGUCUGGUAACGAUGUU | 4 |
| miR-30a | UGUAAACAUCCUCGACUGGAAGCU | 5 |
| miR-10a | UACCCUGUAGAUCCGAAUUUGU | 6 |
| miR-212-3p | UAACAGUCUCCAGUCACGGCCA | 7 |
| miR-15b | UAGCAGCACAUCAUGGUUUACA | 8 |
| miR-660 | UACCCAUUGCAUAUCGGAGUUGU | 9 |
| miR-29a | UAGCACCAUCUGAAAUCGGUU | 10 |
| miR-335-5p | UCAAGAGCAAUAACGAAAAAUG | 11 |
| miR-222 | AGCUACAUCUGGCUACUGGGUCUC | 12 |
| miR-760 | CGGCUCUGGGUCUGUGGGGA | 13 |
| miR-1224-5p | GUGAGGACUCGGGAGGUGGAG | 14 |
| miR-139 | UCUACAGUGCACGUGUCUCCAGU | 15 |
| miR-205 | UCCUUCAUUCCACCGGAGUCUG | 16 |
| miR-9 | UCUUUGGUUAUCUAGCUGUAUGA | 17 |
| miR-93 | CAAAGUGCUGUUCGUGCAGGUAG | 18 |
| miR-379 | UGGUAGACUAUGGAACGUAGG | 19 |
| miR-615 | UCCGAGCCUGGGUCUCCCUCU | 20 |
| miR-92b | UAUUGCACUCGUCCCGGCCUCC | 21 |
| miR-125b | UCCCUGAGACCCUAACUUGUGA | 22 |
| miR-192 | CUGACCUAUGAAUUGACAGCC | 23 |
| miR-149 | UCUGGCUCCGUGUCUUCACUCCC | 24 |
| miR-429 | UAAUACUGUCUGGUAAAACCGU | 25 |
| miR-487b | AAUCGUACAGGGUCAUCCACUU | 26 |
| miR-18a | UAAGGUGCAUCUAGUGCAGAUAG | 27 |
| miR-155 | UUAAUGCUAAUCGUGAUAGGGGU | 28 |
| miR-21 | UAGCUUAUCAGACUGAUGUUGAC | 29 |
| miR-451 | AAACCGUUACCAUUACUGAGUU | 30 |
| miR-486 | UCCUGUACUGAGCUGCCCCGAG | 31 |

Some miRNAs may be made in different DNA locations. Typically, all sequence reads from multiple locations are merged because the miRNA molecule is always the same.

### EXAMPLE 2.

The following is an example of a set of instructions for miRNA-based classification of neuroendocrine neoplasm in a tissue sample. The classification scheme is shown as a flow chart in FIG. 3B. A similar scheme and set of instructions may be developed for liquid (e.g., plasma, serum, and/or other biofluids) samples using methods described above and in Example 3.
1. Expression data for miRNAs in a biological sample were preprocessed (normalized, log2 transformed) and subjected to feature selection and feature evaluation; a subset of 28 miRNAs was selected and loaded in the hierarchical classifier (see Table 3: miR-375, miR-7, miR-10b, miR-200a, miR-93, miR-379, miR-30a, miR-10a, miR-212-3p, miR-15b, miR-660, miR-29a, miR-335-5p, miR-222, miR-760, miR-1224-5p, miR-139, miR-205, miR-9, miR-18a, miR-155, miR-615, miR-92b, miR-125b, miR-192, miR-149, miR-429, miR-487b)
2. Determine Neuroendocrine (NEN) or Non-Neuroendocrine (Non-NEN) using miR-375 and miR-7 in a LDA classifier.
   a. If classified as class ‘NEN’→-NEN (proceed to Step 3)
   b. Otherwise, →Non-NEN.
3. Determine Non-Epithelial or Epithelial using miR-10b and miR-200a as input for the cubic SVM classifier.
   a. If classified as class ‘non-epithelial’→Non-Epithelial (proceed to Step 4)
   b. Otherwise, if classified as class ‘epithelial’→Epithelial (proceed to Step 6)

### For Non-Epithelial:

4. Identify Neuroblastoma (NB) using miR-93 as input for the linear SVM classifier.
   a. If classified as class ‘NB’→NB
   b. Otherwise, if not classified as class ‘NB’→proceed to Step 5
5. Determine Pheochromocytoma (PCC) or Paraganglioma (PGL) using miR-379 and miR-10b as input for the linear SVM classifier.
   a. If classified as class ‘PGL’→PGL
   b. Otherwise, if classified as class ‘PCC’→PCC

### For Epithelial:

6. Identify Parathyroid Adenoma (PTA) using miR-30a as input for the linear SVM classifier.
   a. If classified as class ‘PTA’→PTA
   b. Otherwise, if not classified as class ‘PTA’→proceed to Step 7
7. Identify Pituitary Neuroendocrine Tumour (PitNET) using miR-212-3p and miR-10a as input for the linear SVM classifier.
a. If classified as class ‘PitNET’→PitNET
b. Otherwise, if not classified as class ‘PitNET’→proceed to Step 8
8. Identify Merkel Cell Carcinoma (MCC) using miR-15b and miR-660 as input for the cubic SVM classifier.
   a. If classified as class ‘MCC’→MCC
   b. Otherwise, if not classified as class ‘MCC’→proceed to Step 9
9. Identify Medullary Thyroid Carcinoma (MTC) using miR-29a, miR-335-5p, miR-222 as input for the cubic SVM classifier.
   a. If classified as class ‘MTC’→MTC
   b. Otherwise, if not classified as class ‘MTC’→proceed to Step 10
10. Determine Gastroenteropancreatic (GEP) or Lung neuroendocrine neoplasm using miR-760, miR-1224-5p, miR-139, miR-205, miR-9 as input for the linear SVM classifier.
   a. If classified as class ‘Lung’→Lung (proceed to Step 11)
   b. Otherwise, if classified as class ‘GEP’→GEP (proceed to Step 12)

### For Lung:

11. Determine Carcinoid or Carcinoma using miR-18a and miR-155 as input for the LDA classifier.
   a. If classified as class ‘Carcinoid’→Carcinoid
   20 b. Otherwise, if classified as class ‘Carcinoma’→Carcinoma

### For GEP:

12. Determine Midgut or Non-midgut using miR-615 and miR-92b as input for the linear SVM classifier.
   a. If classified as class ‘Midgut’→Midgut (proceed to step 13)
   b. Otherwise, if classified as class ‘Non-midgut’→Non-midgut (proceed to Step 14)
13. Determine Ileal (INET) or Appendiceal (AppNET) Neuroendocrine Tumour using miR-125b, miR-192, and miR-149 as input for the linear SVM classifier.
   a. If classified as class ‘INET’→INET
   b. Otherwise, if classified as class ‘AppNET’→AppNET
14. Determine Rectal (RNET) or Pancreatic (PanNET) Neuroendocrine Tumour using miR-429 and miR-487b as input for the linear SVM classifier.
   a. If classified as class ‘RNET’→RNET
   b. Otherwise, if classified as class ‘PanNET’→PanNET

### EXAMPLE 3

Described below are methods and materials for miRNA expression profiling, data mining and machine learning, and the development of liquid biopsy tools for (i) discriminating NEN from non-NEN patients and classifying NEN pathological type, and (ii) guiding treatment selection, determining prognosis, and monitoring disease burden.

### Sample Collection and Clinical Information

Plasma, serum, and/or other biofluids are obtained from NEN patients and non-NEN controls using standard processing approaches. Relevant demographic (e.g., age, sex, race) and clinical information (e.g., diagnosis, pathologic type, tumor grade, stage, and treatment response) are obtained.

### RNA Isolation and Quantitation

Total extracellular RNA (exRNA) are obtained from plasma, serum, and/or other biofluids using commercial RNA isolation approaches, such as the QIAGEN miRNeasy Serum/Plasma kit, or non-commercial RNA isolation procedures, such as that described in [17]. Prior to RNA isolation, clinical samples may be “spiked” with RNA calibration markers of known concentration, such as ath-miR-159a (QIAGEN) or a custom cocktail of synthetic oligonucleotide sequences (Max et al. 2018), to assess RNA isolation efficiency and enable miRNA quantitation below.

### Extracellular RNA Sequencing and Sequence Annotation

Extracellular miRNA profiles for each sample are prepared using exRNA sequencing and annotatation [17]. Following exRNA isolation, cDNA libraries are generated through barcoded 3′ oligonucleotide adapter ligation, sample pooling, 5′ adapter ligation, cDNA library preparation, and PCR-based introduction of sequencing primers compatible with Illumina NextSeq and NovaSeq platforms. FASTQ sequence files are annotated through an automated pipeline (rnaworld.rockefeller.edu)[5], yielding sequencing statistics and merged miRNA, miRNA cluster and calibrator read counts. Merged miRNA refers to combined counts of multicopy miRNAs from different genomic locations and miRNA clusters are transcriptional units as defined [10]; the term “miRNA” hereafter refers to merged miRNA data. miRNA content is calculated by comparing sequencing or real-time PCR results below to those obtained for the “spiked in” calibrators of known sequence.

### Data Preprocessing and Filtering

Data preprocessing, filtering, and subsequent analyses are performed in MATLAB

(Mathworks, Inc., Natick, Mass., USA, version R2019a as described [9]). To report miRNA abundance independent of sequencing depth, read counts are normalized against total sequence reads annotated as miRNAs. Sample outliers and batch effects are identified through correlation analyses [11] of miRNA expression profiles and excluded from the final dataset to increase study rigor. Following preprocessing, all non-human miRNAs and human miRNA STAR sequences are excluded from further analyses. Lowly expressed miRNAs and miRNA cistrons may also be filtered using specific thresholds to reduce data dimensionality.

Unsupervised Hierarchical Clustering of Filtered miRNA Expression Profiles

To assess sample grouping, unsupervised hierarchical clustering is performed using log2 transformed normalized read counts of miRNA and miRNA clusters from all preprocessed samples. Euclidean distance is used as the similarity parameter with complete agglomeration hierarchical clustering applied in the heatmap.2 function of the R gplots package (www.rdocumentation.org/packages/gplots/versions/3.0.1.1). Sample grouping is used to guide discovery analyses for classificatory features below.

Discovery Analyses for miRNA-Based NEN Liquid Diagnostics

To identify circulating miRNAs or miRNA clusters that accurately (i) discriminate NEN from non-NEN samples and classify NEN pathological types, and/or (ii) guide treatment selection, determine prognosis, and/or monitor disease, the same classifier construction protocol may be followed as for NEN and non-NEN tissues. First, samples are labeled with relevant clinical information (diagnosis, pathologic type, tumor grade, stage, and treatment response). Next, features that determine the two groups above are identified and ranked using a feature selection algorithm and evaluated using knowledge of miRNA abundance, tissue specificity, and consistent behavior of other miRNA cistron members. Selected miRNAs are carried forward into classifier construction below.

### Construction and Cross-Validation of Decision-Layer and Multilayer Classifiers

A multilayer classifier for (i) discriminating NEN from non NEN samples and classifying NEN pathological types, and (ii) guiding treatment selection, determining prognosis, and/or monitoring disease based in selected miRNAs (through feature selection and evaluation as described above) is constructed and cross-validated. For each decision layer, all available algorithms (n =23) in the MATLAB Classification Learner App may be evaluated using 5-fold cross-validation. In each case, the classification model with highest accuracy, e.g., a support vector machine (SVM) classifier, is used to discriminate between two conditions evaluated at the current case level and comprises the discriminator function for that layer.

Based on these layers, a discriminator function comprising a multilayer classifier is constructed in which miRNA expression profiles are subjected to classification in which (i) they are first classified as NEN or NEN prior to classification to a specific pathological type, and (ii) one or more classifier layers may classify miRNA expression profiles by discriminating between, for example, treatment response conditions.

Performance and transferability of the multilayer classifier may be assessed using t-stochastic neighbor embedding (t-SNE) to visualize sample grouping patterns based on miRNAs selected for classification. Overall classifier accuracy mayl be determined, the impact of miRNA cluster member substitutions on classifier accuracy may be evaluated, and the expression levels of the selected miRNAs inspected.

Classifier Approach may Lead to Simpler miRNA Testing Algorithms

Following construction of a multi-layer hierarchical classifier for determining NEN status and pathologic type, portions of the classifier may be adapted to specific clinical needs. For instance, miR-375 is expected to effectively discriminate NEN from non-NEN samples. Supporting evidence using real-time quantitative real-time polymerase chain reaction measurements of miR-375 in NEN patient plasma is provided in Example 4.

### EXAMPLE 4

A method was developed to measure miR-375 in NEN patient plasma using real-time quantitative polymerase chain reaction. Briefly, 40 mL peripheral blood samples were collected from eight NEN patients, pre- and post-therapy, and five self-reported health controls, and plasma was generated from each sample. Next, 0.1 pmol ath-miR-159a (SEQ ID NO.: 32, UUUGGAUUGAAGGGAGCUCUA) was spiked into 200 μL of plasma from each patient or control. Cell-free total RNA was isolated using the miRNeasy Serum/Plasma Kit (QIAGEN, cat. No. 217184). RNA was measured using BioRad UV spectrophotometry. cDNA was prepared using TaqMan™ MicroRNA Reverse Transcription Kit (ThermoFisher Scientific, cat. no. 4366596) and 10-20 ng total RNA input. miR-375 and ath-miR-159a real-time PCR was performed using TadMan™ Universal Master Mix II, no UNG (ThermoFisher Scientific, cat. no. 4440043) according to the manufacturer's guidelines. PCR cycle threshold (Ct) values for miR-375 and spike-in control were generated on StepOnePlus™ Real-Time PCR System (Applied Biosystems™). miR-375 expressions were generated using the delta-Ct method. Seven treated NEN patients (NETs 1, 4, 7, 16, 17, 19, 21) had plasma miR-375 delta Ct values similar to controls (NETs 22, 23, 24, 25, 26). However, one newly diagnosed and untreated patient with small cell lung carcinoma (NET 2) had a low delta Ct value compared to controls, indicating much higher miR-375 plasma concentration. These findings are consistent with miR-375 being a circulating marker of NEN tumor burden, indicating utility as a biomarker for some smoking-related lung cancers.

The method provides a rapid test for monitoring tumor status, determining prognosis, response to a treatment, and/or effectiveness of a treatment in a subject with a NEN cancer. Moreover, the method provides a rapid diagnostic for any NEN or cancer that has NE component, and may also be used to indicate a state of a NEN pathological type.

All cited publications are incorporated herein by reference in their entirety.

### EQUIVALENTS

While the invention has been described with respect to illustrative embodiments thereof, it will be understood that various changes may be made to the embodiments without departing from the scope of the invention. Accordingly, the described embodiments are to be considered exemplary and the invention is not to be limited thereby.

### REFERENCES

[1] Rindi, G., Klimstra, D. S., Abedi-Ardekani, B., Asa, S. L., Bosman, F. T., Brambilla, E., Busam, K. J., de Krijger, R. R., Dietel, M., El-Naggar, A. K. et al. (2018) A common classification framework for neuroendocrine neoplasms: an International Agency for Research on Cancer (IARC) and World Health Organization (WHO) expert consensus proposal. Mod. Pathol., 31,1770-1786.
[2] Lu, J., Getz, G., Miska, E. A., Alvarez-Saavedra, E., Lamb, J., Peck, D., Sweet-Cordero, A., Ebert, B. L., Mak, R. H., Ferrando, A. A. et al. (2005) MicroRNA expression profiles classify human cancers. Nature, 435,834-838.
[3] Landgraf, P., Rusu, M., Sheridan, R., Sewer, A., lovino, N., Aravin, A., Pfeffer, S., Rice, A., Kamphorst, A. O., Landthaler, M. et al. (2007) A mammalian microRNA expression atlas based on small RNA library sequencing. Cell, 129,1401-1414.
[4] Renwick, N., Cekan, P., Masry, P. A., McGeary, S. E., Miller, J. B., Hafner, M., Li, Z., Mihailovic, A., Morozov, P., Brown, M. et al. (2013) Multicolor microRNA FISH effectively differentiates tumor types. J. Clin. Invest., 123,2694-2702.
[5] Farazi, T. A., Brown, M., Morozov, P., Ten Hoeve, J. J., Ben-Dov, I. Z., Hovestadt, V., Hafner, M., Renwick, N., Mihailovic, A., Wessels, L. F. et al. (2012) Bioinformatic analysis of barcoded cDNA libraries for small RNA profiling by next-generation sequencing. Methods, 58,171-187.
[6] Cheung, I. Y., Farazi, T. A., Ostrovnaya, I., Xu, H., Tran, H., Mihailovic, A., Tuschl, T. and Cheung, N. K. (2014) Deep microRNA sequencing reveals downregulation of miR-29a in neuroblastoma central nervous system metastasis. Genes Chromosomes Cancer, 53,803-814.
[7] Shilo, V., Mor-Yosef Levi, I., Abel, R., Mihailovic, A., Wasserman, G., Naveh-Many, T. and Ben-Dov, I. Z. (2017) Let-7 and microRNA-148 regulate parathyroid hormone levels in secondary hyperparathyroidism. J. Am. Soc. Nephroi., 28,2353-2363.
[8] Mong, E. F., Akat, K. M., Canfield, J., Lockhart, J., VanWye, J., Matar, A., Tsibris, J. C. M., Wu, J. K., Tuschl, T. and Totary-Jain, H. (2018) Modulation of LIN28B/let-7 signaling by propranolol contributes to infantile hemangioma involution. Arterioscler. Thromb. Vasc. Biol., 38,1321-1332.
[9] Panarelli, N., Tyryshkin, K., Wong, J. J. M., Majewski, A., Yang, X., Scognamiglio, T., Kim, M. K., Bogardus, K., Tuschl, T., Chen, Y. T. et al. (2019) Evaluating gastroenteropancreatic neuroendocrine tumors through microRNA sequencing. Endocr. Relat. Cancer, 26,47-57.
[10] Brown, M., Suryawanshi, H., Hafner, M., Farazi, T. A. and Tuschl, T. (2013) Mammalian miRNA curation through next-generation sequencing. Front. Genet., 4,145.
[11] Ren, R., Tyryshkin, K., Graham, C. H., Koti, M. and Siemens, D. R. (2017) Comprehensive immune transcriptomic analysis in bladder cancer reveals subtype specific immune gene expression patterns of prognostic relevance. Oncotarget, 8,70982-71001.
[12] Duan, K. and Mete, O. (2016) Algorithmic approach to neuroendocrine tumors in targeted biopsies: practical applications of immunohistochemical markers. Cancer Cytopathol., 124,871-884.
[13] Uccella, S., La Rosa, S., Volante, M. and Papotti, M. (2018) Immunohistochemical biomarkers of gastrointestinal, pancreatic, pulmonary, and thymic neuroendocrine neoplasms. Endocr. Pathol., 29,150-168.
[14] Hafner, M., Renwick, N., Farazi, T. A., Mihailovic, A., Pena, J. T. and Tuschl, T. (2012) Barcoded cDNA library preparation for small RNA profiling by next-generation sequencing. Methods, 58, 164-170.
[15] Ludwig, N., Leidinger, P., Becker, K., Backes, C., Fehlmann, T., Pallasch, C., Rheinheimer, S., Meder, B., Stahler, C., Meese,E. et al. (2016) Distribution of miRNA expression across human tissues. Nucleic Acids Res., 44, 3865-3877.
[16] La Rosa, S., Sessa, F. and Uccella, S. (2016) Mixed neuroendocrine-nonneuroendocrine neoplasms (MiNENs): unifying the concept of a heterogeneous group of neoplasms. Endocr. Pathol., 27, 284-311.
[17] Max, K. E. A., Bertram, K., Akat, K. M., Bogardus, K. A., Li, J., Morozov, P., Ben-Dov, I. Z., Li, X., Weiss, Z. R., Azizian, A., Sopeyin, A., Diacovo, T. G., Adamidi, C., Williams, Z., Tuschl, T. (2018) Human plasma and serum extracellular small RNA reference profiles and their clinical utility. Proc. Natl. Acad. Sci. USA., 115(23), E5334-E5343. doi: 10.1073/pnas.1714397115.

1. A method for classifying cancer, comprising:
obtaining data relating to expression levels of at least two selected miRNAs (miRs) in a biological sample from a subject;
using a processor to subject the data to a discriminator function;
wherein the discriminator function uses at least one selected feature in the data, the at least one selected feature being related to at least a first condition of interest;
wherein the discriminator function comprises at least one trained classifier that classifies the cancer according to at least a first condition of interest based the at least one selected feature in the data.

2. The method of claim 1, wherein the discriminator function comprises at least one classifier trained to use at least two selected features comprising expression levels of miR-375 and miR-7;
wherein the at least one trained classifier classifies the cancer as a neuroendocrine neoplasm (NEN) or a non-neuroendocrine neoplasm (non-NEN).

3. The method of claim 2, wherein the discriminator function comprises at least one classifier trained to use at least two selected features comprising expression levels of miR-200a and miR-10b;
wherein the at least one trained classifier classifies the cancer according to a condition of interest as an epithelial NEN based on elevated expression of miR-200a and reduced expression of miR-10b, otherwise the at least one trained classifier classifies the cancer as a non-NEN.

4. The method of claim 3, wherein the discriminator function comprises at least one classifier trained to use at least a selected feature comprising an expression level of miR-30a;
wherein the at least one trained classifier classifies the NEN according to a condition of interest as parathyroid adenoma (PTA) based on elevated expression of miR-30a, otherwise the at least one trained classifier classifies the epithelial NEN as non-PTA.

5. The method of claim 4, wherein the discriminator function comprises at least one classifier trained to use at least two selected features comprising expression levels of miR-10a and miR-212-3p;
wherein the at least one trained classifier classifies the epithelial non-PTA NEN according to a condition of interest as pituitary adenoma (PitNET) based on reduced expression of miR-10a and elevated expression of miR-212-3p, otherwise the at least one trained classifier classifies the epithelial neuroendocrine neoplasm as non-PitNET.

6. The method of claim 5, wherein the discriminator function comprises at least one classifier trained to use at least two selected features comprising expression levels of miR-15b and miR-660;
wherein the at least one trained classifier classifies the epithelial non-PitNET NEN according to a condition of interest as Merkel cell carcinoma (MCC) based on elevated expression of miR-15b and miR-660, otherwise the at least one trained classifier classifies the epithelial NEN as non-MCC.

7. The method of claim 6, wherein the discriminator function comprises at least one classifier trained to use at least three selected features comprising expression levels of miR-29a, miR-335-5p, and miR-222;
wherein the at least one trained classifier classifies the epithelial non- MCC NEN according to a condition of interest as medullary thyroid carcinoma (MTC) based on elevated expression levels of miR-29a and miR-222 and reduced expression level of miR-335-5p, otherwise the at least one trained classifier classifies the epithelial NEN as non-MTC.

8. The method of claim 7, wherein the discriminator function comprises at least one classifier trained to use at least five selected features comprising expression levels of miR-760, miR-1224-5p, miR-139, miR-205, and miR-9;
wherein the at least one trained classifier classifies the epithelial non-MTC NEN according to a condition of interest as gastroenteropancreatic (GEP) neoplasm based on elevated expression levels of miR-760, miR-1224-5p, miR-139, and miR-205 and reduced expression level of miR-9, otherwise the at least one trained classifier classifies the epithelial non-MTC NEN as lung neuroendocrine neoplasm.

9. The method of claim 8, wherein the discriminator function comprises at least one classifier trained to use at least two selected features comprising expression levels of miR-615, and miR-92b;
wherein the at least one trained classifier classifies the GEP neoplasm according to a condition of interest as midgut based on elevated expression levels of miR-615 and reduced expression level of miR-92b, otherwise the at least one trained classifier classifies the GEP neoplasm as non-midgut.

10. The method of claim 9, wherein the discriminator function comprises at least one classifier trained to use at least three selected features comprising expression levels of miR-149, miR-192, and miR-125b;
wherein the at least one trained classifier classifies the midgut neoplasm according to a condition of interest as ileal neoplasm (INET) based on elevated expression level of miR-192 and reduced expression level of miR-125b and miR-149; otherwise the at least one trained classifier classifies the midgut neoplasm as appendiceal neoplasm (AppNET).

11. The method of claim 9, wherein the discriminator function comprises at least one classifier trained to use at least two selected features comprising expression levels of miR-487b, and miR-429;
wherein the at least one trained classifier classifies the midgut neoplasm according to a condition of interest as rectal neoplasm (RNET) based on elevated expression level of miR-429 and reduced expression level of miR-487b; otherwise the at least one trained classifier classifies the midgut neoplasm as pancreatic neoplasm (PanNET).

12. The method of claim 8, wherein the discriminator function comprises at least one classifier trained to use at least two selected features comprising expression levels of miR-18a and miR-155;
wherein the at least one trained classifier classifies the lung neoplasm according to a condition of interest as carcinoid (TC/AC) based on reduced expression level of miR-18a and miR-155; otherwise the at least one trained classifier classifies the lung neoplasm as carcinoma (SCLC/LCNEC).

13. The method of claim 3, wherein the discriminator function comprises at least one classifier trained to use at least a selected feature comprising expression level of miR-93;
wherein the at least one trained classifier classifies the non-epithelial NEN according to a condition of interest as neuroblastoma (NB) based on elevated expression of miR-93; otherwise the at least one trained classifier classifies the non-epithelial NEN as non-NB.

14. The method of claim 13, wherein the discriminator function comprises at least one classifier trained to use at least two selected features comprising expression levels of miR-10b and miR-379;
wherein the at least one trained classifier classifies the non-NB neoplasm according to a condition of interest as pheochromocytoma (PCC) based on elevated expression level of miR-10b and reduced expression level of miR-379, or the at least one trained classifier classifies the non-epithelial NEN as paraganglioma (PGL) based on reduced expression level of miR-10b and elevated expression level of miR-379.

15. The method of claim 1, comprising preprocessing the data prior to subjecting the data to a discriminator function.

16. A method for evaluating a NEN cancer in a subject, comprising:
measuring expression levels of at least two miRNAs in a biological sample obtained from the subject;
wherein at least a first miRNA is miR-375;
determining an expression level of miR-375 relative to an expression level of at least a second miRNA in the biological sample;
determining expression levels of the at least two miRNAs in biological samples obtained from subjects without cancer;
wherein an elevated expression level of miR-375 relative to the expression level of the at least a second miRNA is used to determine NEN cancer status in the subject.

17. The method of claim 16, comprising measuring expression levels of at least two miRNAs in first and second biological samples obtained from the subject at first and second instants in time;
wherein a change in expression level of miR-375 relative to the expression level of the second miRNA across the first and second biological samples is used to determine NEN cancer status in the subject.

18. The method of claim 16 or 17, wherein the at least a second miRNA is added to the biological sample(s).

19. The method of claim 18, wherein the at least a second miRNA is miR-159a.

20. The method of claim 16 or 17, wherein the at least a second miRNA is at least one of miR-7, miR-451, miR-486, and miR-21.

21. The method of any one of claims 16-20, comprising measuring expression levels using real-time quantitative polymerase chain reaction and comparing expression levels based on the delta-Ct values.

22. The method of any one of claims 16-21, wherein the biological sample(s) comprise tissue sample(s).

23. The method of any one of claims 16-21, wherein the biological sample(s) comprise liquid sample(s).

24. The method of any one of claims 16-23, wherein determining NEN cancer status comprises one or more of differentiating between NEN and non-NEN, diagnosing a NEN pathological type, monitoring disease state and subject condition, determining prognosis, evaluating response to a treatment, evaluating effectiveness of a treatment.

25. Non-transitory computer readable media for use with a processor, the computer readable media having stored thereon instructions that direct the processor to:
receive data; and
construct a discriminator function by:
(i) subjecting the data to at least one feature selection, ranking, and evaluation algorithm, wherein at least one feature related to at least a first condition of interest is selected;
(ii) using the at least one selected feature to train at least one classifier, wherein the at least one classifier outputs a first classification result for the at least a first condition of interest based on the at least one feature; and
(iii) storing parameters of the at least one classifier trained for the at least a first condition of interest;
wherein a discriminator function comprising the at least one classifier trained for the at least a first condition of interest is constructed.

26. The non-transitory computer readable media of claim 25, wherein the instructions direct the processor to:
repeat steps (i) to (iii) for a plurality of conditions of interest and store parameters of at least one classifier trained for each of the plurality of conditions of interest;
wherein a discriminator function comprising a plurality of trained classifiers, wherein at least one classifier is trained for each of the plurality of conditions of interest, is constructed.

27. The non-transitory computer readable media of claim 25, wherein the data are selected from omics data, imaging data, and electromagnetic spectra data.
